(19) United States
(12) Patent Application Publication
(10) Pub. No.: US 2010/0034835 A1
(43) Pub. Date: Feb. 11, 2010

(54) USE OF INHIBITORS OF LEUKOTRIENE B4 RECEPTOR BLT2 FOR TREATING ASTHMA

(75) Inventors: Jae-Hong Kim, Gyeonggi-do (KR); Kyung-Jin Cho, Busan (KR); Min-Hyuk Yoo, Seoul (KR)

Correspondence Address: THE NATH LAW GROUP, 112 South West Street, Alexandria, VA, 22314, US

(73) Assignee: Korea University Industrial & Academic Collaboration foundation, Seoul (KR)

(21) Appl. No.: 12/450,342

(22) PCT Filed: Mar. 24, 2008
(86) PCT No.: PCT/KR2008/001650

§ 371 Date: Sep. 22, 2009

Related U.S. Application Data
(60) Provisional application No. 60/896,501, filed on Mar. 23, 2007.
(60) Provisional application No. 60/896,502, filed on Mar. 23, 2007.

(51) Int. Cl. A61K 39/395; A61K 31/41; A61K 48/00; A61P 11/06; C12Q 1/68; C12Q 1/02; C07H 21/04

(52) U.S. Cl. 424/175.1; 514/381; 514/44 A; 435/6; 435/29; 536/24.33; 536/24.3

(57) ABSTRACT

The present invention relates to a new use of inhibitors of leukotriene B4 receptor BLT2 for treating asthma. More particularly, the present invention relates to a pharmaceutical composition for treating asthma comprising BLT2 inhibitors and a method for treating asthma using BLT2 inhibitors.

The present inventors investigated the role of BLT2 in the pathogenesis of asthma using a murine model and demonstrated that BLT2 plays a critical role in the development of AHR and airway inflammation by employing BLT2 inhibitors, such as antisense oligonucleotide. Therefore, the BLT 2 inhibitors according to the present invention can be effectively used as a therapeutic composition for treating asthma.

### TECHNICAL FIELD

The present invention relates to a new use of inhibitors of leukotriene B4 receptor BLT2 for treating asthma. More particularly, the present invention relates to a pharmaceutical composition for treating asthma comprising BLT2 inhibitors and a method for treating asthma using BLT2 inhibitors.

### BACKGROUND ART

Leukotriene B4 (LTB4) is a key mediator of inflammatory processes, immune responses, and host defenses against infection (1-4). It stimulates chemotaxis, degranulation, release of lysosomal enzymes, and the production of reactive oxygen species (ROS) (5-7). In fact, LTB4 is one of the most potent chemoattractants known, acting mainly on granulocytes and monocytes (8, 9). Recently, it was also shown to be a chemoattractant for effector CD4+ and CD8+ T lymphocytes, recruiting them to sites of acute inflammation (10-15). It also promotes cell adhesion to vascular endothelial cells and transmigration (8, 16), which amplifies inflammatory early responses. Although LTB4-induced leukocyte recruitment is thought to play a protective role in host defense against various pathogens, it is also involved in a number of human inflammatory diseases such as asthma (17-20), a disease of chronic airway inflammation characterized by eosinophilic infiltration, mucus hypersecretion, and airway hyperresponsiveness (AHR). Thus, a significantly increased level of LTB4 is detected in the airways of patients with asthma and also in experimental models of asthma (20).

LTB4 produces its biological effects via specific G protein-coupled receptors known as BLT1 and BLT2 (21-24). To date, most studies of LTB4 receptors have focused on the high-affinity LTB4 receptor, BLT1, expressed exclusively in leukocytes, especially its role in inflammatory responses (22). For example, early recruitment of neutrophils and eosinophils into the airways in response to allergen inhalation is reduced in BLT1-deficient mice (8, 25), suggesting a role of BLT1 in the chemotaxis of granulocytes in allergic asthma. In addition, BLT1 is essential for the allergen-mediated early recruitment of CD4+ and CD8+ T cells into the lung airways and the development of allergen-induced AHR and inflammation under certain experimental conditions (26, 27). In contrast to BLT1, BLT2 has a low affinity for LTB4 and is expressed in a wide variety of tissues, with highest levels in the spleen, leukocytes and ovary (23).

In the present invention, we investigated the role of BLT2 in the pathogenesis of asthma using a murine model. We demonstrate, by employing antisense to block endogenous BLT2 expression, that BLT2 plays a critical role in the development of AHR and airway inflammation. In addition, we present evidence that BLT2 causes asthmatic symptoms by elevating ROS generation and subsequent NF-κB activation. Furthermore, immunohistochemical analysis of clinical asthma samples revealed a significant elevation of expression of BLT2 mainly in the airway epithelial layers as well as in the microvascular endothelium, which is similar to the pattern observed in the murine model of asthma.

Throughout this application, several patents and publications are referenced or cited in parentheses. The disclosure of these patents and publications is incorporated into this application in order to more fully describe this invention and the state of the art to which this invention pertains.

### DISCLOSURE OF INVENTION

The object of the present invention is to provide a new use of BLT2 inhibitors for the manufacture of a medicament for the treatment of asthma.

Further, another object of the present invention is to provide a pharmaceutical composition for the treatment of asthma comprising BLT2 inhibitors as an active ingredient.

Further, another object of the present invention is to provide a method for treating a patient with asthma, which comprises administering of BLT2 inhibitors to the patient.

Further, another object of the present invention is to provide a method for screening a substance for treating asthma, which comprises determining whether to reduce the expression or signaling level of BLT2.

Further, another object of the present invention is to provide a kit for detecting asthma, which comprises a primer or probe for detecting BLT2 gene or an antibody for detecting BLT2 protein.

Further, another object of the present invention is to provide a new use of Rac inhibitors for the manufacture of a medicament for the treatment of asthma.

Other objects and advantage of the present invention will become apparent from the detailed description to follow taken in conjugation with the appended claims and drawings.

BLT2 is a low-affinity receptor for leukotriene B4 (LTB4), a potent lipid mediator of inflammation generated from arachidonic acid via the 5-lipoxygenase pathway. Unlike BLT1, a high-affinity receptor for LTB4, no physiological role has yet been identified for BLT2, especially with regard to the pathogenesis of asthma. We have used a murine model of allergic asthma to evaluate the role of BLT2 in ovalbumin-induced airway inflammation and airway hyperresponsiveness (AHR). The levels of BLT2 mRNA and its ligand LTB4 in the lung airway were highly elevated after OVA challenge, and downregulation of BLT2 with antisense BLT2 oligonucleotides markedly attenuated the airway inflammation and AHR, suggesting a role of BLT2 in the asthmatic response. Further analysis aimed at identifying mediators downstream of BLT2 revealed that BLT2 activation led to elevation of reactive oxygen species (ROS) and subsequent activation of NF-κB, thus inducing the expression of VCAM-1 that is known to be involved in eosinophil infiltration into lung airway. Together our findings suggest that BLT2 plays a pivotal role in the pathogenesis of asthma, acting through a ‘ROS-NF-κB’-linked signaling pathway. Finally, immunohistochemical assay of clinical subjects demonstrated that BLT2 expression was high in the airway epithelial layers as well as the microvascular endothelium, as in the murine model of asthma.

According to one aspect of the present invention, there is provided a use of a substance that inhibits the expression or intracellular signaling of BLT2 for the manufacture of a medicament for the treatment of asthma. In this specification, the phrase “inhibit(s) the expression of BLT2” means to inhibit any step among the transcription, mRNA processing, translation, translocation, and maturation of BLT2, and the phrase “inhibit(s) the intracellular signaling of BLT2” means to inhibit any step among the binding of LTB4 to BLT2, the activation of BLT2 and its intracellular signaling pathway to induce asthma.

The nucleotide sequence of human BLT2 gene is available at the NCBI (NM_{—}019839) and denoted as SEQ ID NO: 1 in this specification. The BLT2 gene has 2 kinds of CDS form, long form CDS (1618-2787) and short form CDS (1711-2787), of which base sequences are denoted as SEQ ID NO: 2 and SEQ ID NO: 4. The amino acid sequence of the long form BLT2 protein is available at the NCBI (NM_{—}019839) and denoted as SEQ ID NO: 3. The amino acid sequence of the long form BLT2 protein is available at the NCBI (AB029892) and denoted as SEQ ID NO: 5.

In a preferred embodiment, the substance may be a compound that binds to BLT2 and inhibits the intracellular signaling of BLT2. The compound is also referred to as BLT2 antagonist, which means a compound that antagonizes an action of LTB4 on BLT2. The compound can be screened according to the present screening method from the commercially available chemical DB.

In a preferred embodiment, the compound may be LY255283 (1-[5-ethyl-2-hydroxy-4-[[6-methyl-6-(1H-tetrazol-5-yl)heptyl]oxy]phenyl]-ethanone). FIG. 1a shows a chemical structure of LY255283. LY255283 is a competitive antagonist of the BLT2 receptor. LY255283 have been known to inhibit eosinophil chemotaxis by 80% at a concentration of 10 μM, and inhibits the binding of radiolabeled LTB4 to eosinophil membranes with an IC50 of 260 nM [Ann N Y Acad Sci 629 274-287 (1991)]. Also, LY255283 have been known to be a novel leukotriene B4 receptor antagonist, which limits activation of neutrophils and prevents acute lung injury induced by endotoxin in pigs [Surgery. 1993 August; 114(2):191-8]. However, the anti-asthma activity of LY25583 was revealed by the present inventors for the first time.

In a preferred embodiment, the substance may be an antibody to BLT2 that inhibits the intracellular signaling of BLT2. The antibody binds to BLT2 competitively with LTB4, so that can inhibit the intracellular signaling of BLT2. The antibody can be produced according to the conventional methods for producing polyclonal or monoclonal antibody by using BLT2 or its fragment as an antigen.

In a preferred embodiment, the substance may be an antisense or siRNA oligonucleotide that inhibits the expression of BLT2. The antisense or siRNA oligonucleotide has a base sequence complementary to the nucleotide sequence of BLT2 mRNA as set forth in SEQ ID NO: 2.

The term “antisense oligonucleotide” used herein is intended to refer to nucleic acids, preferably, DNA, RNA or its derivatives, that are complementary to the base sequences of a target mRNA, characterized in that they binds to the target mRNA and interfere its translation to protein. The antisense oligonucleotide of this invention means DNA or RNA sequences complementary and binding to BLT2 mRNA, that are able to inhibit translation, translocation, maturation or other biological functions of BLT2 mRNA. The antisense nucleic acid is 6-100, preferably, 8-60, more preferably, 10-40 nucleotides in length.

The antisense oligonucleotide may comprise at lease one modification in its base, sugar or backbone for its higher inhibition efficacy (De Mesmaeker et al., Curr Opin Struct Biol., 5(3):343-55 (1995)). The modified nucleic acid backbone comprises phosphorothioate, phosphotriester, methyl phosphonate, short chain alkyl or cycloalkyl intersugar linkages or short chain heteroatomic or heterocyclic intersugar linkages. The antisense oligonucleotide may also contain one or more substituted sugar moieties. The antisense nucleic acid may include one or more modified bases, for example, hypoxanthine, 6-methyladenine, 5-me pyrimidines (particularly, 5-methylcytosine), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N⁶(6-aminohexyl)adenine and 2,6-diaminopurine. Another modification of the oligonucleotides of the invention involves chemically linking to the oligonucleotide one or more moieties or conjugates which enhance the activity or cellular uptake of the oligonucleotide. Such moieties include but are not limited to lipid moieties such as a cholesterol moiety, a cholesteryl moiety (Letsinger et al., Proc. Natl. Acad. Sci. USA, 86:6553 (1989)), cholic acid (Manoharan et al. Bioorg. Med. Chem. Let., 4:1053 (1994)), a thioether, e.g., hexyl-5-tritylthiol (Manoharan et al. Ann. N.Y. Acad. Sci., 660:306 (1992); Manoharan et al. Bioorg. Med. Chem. Let., 3: 2765 (1993)), a thiocholesterol (Oberhauser et al., Nucl. Acids Res., 20:533 (1992)), an aliphatic chain, e.g., dodecandiol or undecyl residues (Saison-Behmoaras et al. EMBO J., 10:111 (1991); Kabanov et al. FEBS Lett., 259:327 (1990); Svinarchuk et al. Biochimie, 75:49 (1993), a phospholipid, e.g., di-hexadecyl-rac-glycerol or triethylammonium 1,2-di-O-hexadecyl-rac-glycero-3-H-phosphonate (Manoharan et al. Tetrahedron Lett., 36:3651 (1995); Shea et al. Nucl. Acids Res., 18:3777 (1990)), a polyamine or a polyethylene glycol chain (Manoharan et al. Nucleosides & Nucleotides, 14:969 (1995)), or adamantane acetic acid (Manoharan et al. Tetrahedron Lett., 36: 3651 (1995)). Oligonucleotides comprising lipophilic moieties, and methods for preparing such oligonucleotides are known in the art, for example, U.S. Pat. Nos. 5,138,045, 5,218,105 and 5,459,255. The modifications described above enhance stability against nuclease degradation and increase affinity of the antisense oligonucleotide toward its target mRNA.

The antisense molecule is conventionally synthesized in vitro and then transmitted to cells. In addition, it is intracellularly produced by transcription from foreign sequence. In vitro synthesis involves RNA polymerase 1. In vivo transcription for preparing antisense RNA uses vector having origin of recognition region (MCS) in opposite orientation. The antisense RNA preferably comprises a translation stop codon for inhibiting translation to peptide.

According to a preferred embodiment, the antisense oligonucleotide may have a base sequence of SEQ ID NO: 6, which is complementary to the target region (1738-1752) of SEQ ID NO: 2.

According to a preferred embodiment, the siRNA oligonucleotide may have a sense sequence of SEQ ID NO: 7 and an antisense sequence of SEQ ID NO: 8, which is complementary to the target region (1705-1724) of SEQ ID NO: 2.

The term “siRNA” used herein refers to a nucleic acid molecule mediating RNA interference or gene silencing (see WO 00/44895, WO 01/36646, WO 99/32619, WO 01/29058, WO 99/07409 and WO 00/44914). The siRNA to inhibit expression of a target gene provides effective gene knock-down method or gene therapy method. It was been first in plants, insects, Drosophila melanogaster and parasites and recently has been used for mammalian cell researches.

The siRNA molecule of this invention may consist of a sense RNA strand (having sequence corresponding to BLT2 mRNA) and an antisense RNA strand (having sequence complementary to BLT2 mRNA) and form a duplex structure. Alternatively, the siRNA molecule of this invention may have a single strand structure comprising self-complementary sense and antisense strands.

The siRNA of this invention is not restricted to a RNA duplex of which two strands are completely paired and may comprise non-paired portion such as mismatched portion with non-complementary bases and bulge with no opposite bases. The overall length of the siRNA is 10-100 nucleotides, preferably, 15-80 nucleotides, and more preferably, 20-70 nucleotides.

The siRNA may comprise either blunt or cohesive end so long as it enables to silent the BLT2 expression due to RNAi effect. The cohesive end may be prepared in 3′-end overhanging structure or 5′-end overhanging structure.

The siRNA may be constructed by inserting a short nucleotide sequence (e.g., about 5-15 nt) between self-complementary sense and antisense strands. The siRNA expressed forms a hairpin structure by intramolecular hybridization, resulting in the formation of stem-and-loop structure. The stem-and-loop structure is processed in vitro or in vivo to generate active siRNA molecule mediating RNAi.

In the preferred embodiment, the substance may be a compound that inhibits the upstream or downstream signaling pathway of BLT2.

In the preferred embodiment, the asthma may be characterized by that BLT2 protein is over-expressed in the lung airway. The present inventors have found that BLT2 protein and its ligand LTB4 were over-expressed in the lung airway after OVA challenge, and downregulation of BLT2 with antisense BLT2 oligonucleotides markedly attenuated the airway inflammation and AHR. Therefore, any anti-asthma therapy strategy based on the inhibition of BLT2 overexpression is claimed as the present invention.

In the preferred embodiment, the over-expression, i.e. activation of BLT2 may cause asthmatic symptoms by elevating ROS generation and subsequent NF-κB activation. The present inventors demonstrated that the BLT2 activation led to elevation of reactive oxygen species (ROS) and subsequent activation of NF-κB, thus inducing the expression of VCAM-1 that is known to be involved in eosinophil infiltration into lung airway.

In the preferred embodiment, the treatment of asthma may be accomplished by reducing eosinophil infiltration into lung airway, airway inflammation and airway hyperresponsiveness (AHR). Therefore, any use of BLT2 inhibitors as a therapeutic composition against asthma is claimed in the present invention.

According to another aspect of the present invention, there is provided a use of a combination of (a) a substance that inhibits the expression or intracellular signaling of BLT2, and (b) other anti-asthma drugs for the manufacture of a medicament for the treatment of asthma.

According to another aspect of the present invention, there is provided a use of a substance that inhibits the expression or activity of Rac for the manufacture of a medicament for the treatment of asthma. In this specification, the phrase “inhibit(s) the expression of Rac” means to inhibit any step among the transcription, mRNA processing, translation, translocation, and maturation of Rac, and the phrase “inhibit(s) the activity of Rac” means to inhibit any step among the GTPase activity of Rac and its intracellular signaling pathway to induce asthma.

Rac, a member of Rho family GTPases, mediates various cellular responses such as actin polymerization, cell proliferation, cPLA2 activation, and generation of reactive oxygen species (ROS). We have used a mouse model system for asthma to determine the role of Rac1 on allergen-induced bronchial inflammation and airway hyperresponsiveness (AHR). Rac1 activity is dramatically stimulated after allergen challenge and administration of antisense oligomers to Rac1 remarkably reduced bronchial inflammation and AHR. In a further study to determine the signaling mechanism by which Rac1 mediates asthmatic inflammation and AHR, we found out that Rac1 is responsible for the NFkB activation critically implicated in the transcription of various inflammatory genes such as VCAM-1. Additionally, Rac1 was shown to mediate the activation of cPLA2, which catalyzes the hydrolysis of membrane phospholipids leading to the release of arachidonic acid (AA) and subsequently eicosanoids such as leukotrienes (LTs). Together, these findings indicate that Rac1 is critically involved in the pathogenesis of the bronchial asthma. In the preferred embodiment, the substance may be an antisense or siRNA oligonucleotide that inhibits the expression of Rac. The antisense or siRNA oligonucleotide has a base sequence complementary to the nucleotide sequence of Rac mRNA as set forth in SEQ ID NO: 13. The sequence of mRNA or CDS of human Rac gene is available at the NCBI (gi:156071511) and its deduced amino acid sequence is denoted as SEQ ID NO: 14.

According to another aspect of the present invention, there is provided a pharmaceutical composition for the treatment of asthma, which comprises a substance that inhibits the expression or intracellular signaling of BLT2 as an active ingredient. In the pharmaceutical composition of the present invention, the substance may be chemical compounds, peptides, antibody proteins, nucleotides, antisense oligonucleotides, siRNA oligonucleotides or extract of natural source. The present pharmaceutical composition may comprise a pharmaceutically acceptable carrier in addition.

According to another aspect of the present invention, there is provided a pharmaceutical composition for the treatment of asthma, which comprises a substance that inhibits the expression or activity of Rac as an active ingredient. In the pharmaceutical composition of the present invention, the substance may be chemical compounds, peptides, antibody proteins, nucleotides, antisense oligonucleotides, siRNA oligonucleotides or extract of natural source. The present pharmaceutical composition may comprise a pharmaceutically acceptable carrier in addition.

According to another aspect of the present invention, there is provided a method for treating a patient with asthma, which comprises administering a therapeutically effective amount of a substance that inhibits the expression or intracellular signaling of BLT2 to the patient.

According to another aspect of the present invention, there is provided a method for treating a patient with asthma, which comprises administering a therapeutically effective amount of a substance that inhibits the expression or activity of Rac to the patient.

According to another aspect of the present invention, there is provided a method for screening a substance for treating asthma, which comprises the steps of:

(a) contacting the substance to be analyzed to a cell containing BLT2 gene or protein; and,
(b) measuring the expression or intracellular signaling level of BLT2, wherein if the the expression or intracellular signaling level of BLT2 is down-regulated, the substance is determined to have a potency to treat asthma.

According to the present method, the cell containing the BLT2 gene or protein can be easily prepared by obtaining cells containing their original BLT2 gene or by transfecting cells with a foreign BLT 2 gene. The cells containing the BLT2 gene or protein are first contacted to substances to be analyzed. The term “substance” used herein in conjunction with the present screening method refers to a material tested in the present method for analyzing the influence on the expression level of the BLT2 gene, the amount of the BLT2 protein or the intracellular signaling level of the BLT2 receptor. The substance includes chemical compounds, peptides, antibody proteins, nucleotides, antisense-RNA, siRNA (small interference RNA) and extract of natural source, but not limited to.

Afterwards, the expression level of the BLT2 gene, the amount of the BLT2 protein or the intracellular signaling level of the BLT2 receptor in cells is measured. Where the expression level of the BLT2 gene, the amount of the BLT2 protein or the intracellular signaling level of the BLT2 receptor is measured to be down-regulated, the substance is determined to be a candidate to treat asthma.

The measurement of the expression level of the BLT2 gene could be carried out by a variety of methods known in the art. For example, RT-PCR (Sambrook et al., Molecular Cloning. A Laboratory Manual, 3rd ed. Cold Spring Harbor Press (2001)), Northern blotting (Peter B. Kaufma et al., Molecular and Cellular Methods in Biology and Medicine, 102-108, CRC press), hybridization using cDNA microarray (Sambrook et al., Molecular Cloning. A Laboratory Manual, 3rd ed. Cold Spring Harbor Press (2001)) and in situ hybridization (Sambrook et al., Molecular Cloning. A Laboratory Manual, 3rd ed. Cold Spring Harbor Press (2001)) may be used.

Where the expression level of the BLT2 gene is analyzed by RT-PCT, total RNA is first isolated from cells treated with a substance to be analyzed and a first cDNA strand is then synthesized using oligo dT primer and reverse transcriptase. Then, PCR amplifications are performed using the first cDNA strand as templates and a BLT2-specific primer set. Finally, the PCR amplified products are resolved by electrophoresis and bands are analyzed for assessing the expression level of the BLT2 gene.

The amount of the BLT2 protein may be determined by various immunoassays known in the art. For example, radioimmunoassay, radioimmunoprecipitation, enzyme-linked immunosorbent assay (ELISA), capture-ELISA, inhibition or competition assay and sandwich assay are used for analyzing the amount of the BLT2 protein.

The intracellular signaling level of the BLT2 receptor may be determined by monitoring an event induced by LTB4, e.g., monitoring the rise of the intracellular calcium concentration as described in example using BLT2-expressing cells etc. (e.g., BLT2 overexpressing cells etc.). For example, if the substance reduces the intracellular calcium concentration by LTB4 in BLT2-expressing cells, it can be judged as BLT2 antagonist.

According to another aspect of the present invention, there is provided a method for screening a substance for treating asthma, which comprises the steps of:

(a) contacting the substance to be analyzed to a cell containing Rac gene or protein; and,
(b) measuring the expression or activity level of Rac, wherein if the expression or activity level of Rac is down-regulated, the substance is determined to have a potency to treat asthma.

According to another aspect of the present invention, there is provided a kit for detecting asthma, which comprises a primer or probe having a base sequence complementary to the base sequence of BLT2 gene as set forth in SEQ ID NO: 2. Therefore, any methodology or kit developed based on the information that BLT2 overexpression is detected in the lung airway of a patient with asthma may be included in the present invention.

The probes or primers used in the present kit has a complementary sequence to the nucleotide sequence of the BLT2 gene. The term “complementary” with reference to sequence used herein refers to a sequence having complementarity to the extent that the sequence anneals or hybridizes specifically with the nucleotide sequence of the BLT2 gene under certain annealing or hybridization conditions. In this regard, the term “complementary” used herein has different meaning from the term “perfectly complementary”. The probes or primers used in the present invention can be one or more mismatch, so long as such mismatches are not sufficient to completely preclude specific annealing or hybridization to the BLT2 gene.

As used herein the term “probe” means a linear oligomer of natural or modified monomers or linkages, including deoxyribonucleotides and ribonucleotides, capable of specifically binding to a target polynucleotide. The probe may be naturally occurring or artificially synthesized. The probe is preferably single stranded. Preferably, the probes used in the present invention are oligodeoxyribonucleotides. The probe of this invention can be comprised of naturally occurring dNMP (i.e., dAMP, dGM, dCMP and dTMP), modified nucleotide, or non-natural nucleotide. The primer can also include ribonucleotides. For instance, the probes of this invention may include nucleotides with backbone modifications such as peptide nucleic acid (PNA) (M. Egholm et al., Nature, 365:566-568 (1993)), phosphorothioate DNA, phosphorodithioate DNA, phosphoramidate DNA, amide-linked DNA, MMI-linked DNA, 2′-O-methyl RNA, alpha-DNA and methylphosphonate DNA, nucleotides with sugar modifications such as 2′-O-methyl RNA, 2′-fluoro RNA, 2′-amino RNA, 2′-O-alkyl DNA, 2′-O-allyl DNA, 2′-O-alkynyl DNA, hexose DNA, pyranosyl RNA, and anhydrohexitol DNA, and nucleotides having base modifications such as C-5 substituted pyrimidines (substituents including fluoro-, bromo-, chloro-, iodo-, methyl-, ethyl-, vinyl-, formyl-, ethynyl-, propynyl-, alkynyl-, thiazolyl-, imidazolyl-, pyridyl-), 7-deazapurines with C-7 substituents (substituents including fluoro-, bromo-, chloro-, iodo-, methyl-, ethyl-, vinyl-, formyl-, alkynyl-, alkenyl-, thiazolyl-, imidazolyl-, pyridyl-), inosine, and diaminopurine.

The term “primer” as used herein refers to an oligonucleotide, which is capable of acting as a point of initiation of synthesis when placed under conditions in which synthesis of primer extension product which is complementary to a nucleic acid strand (template) is induced, i.e., in the presence of nucleotides and an agent for polymerization, such as DNA polymerase, and at a suitable temperature and pH. The suitable length of primers will depend on many factors, including temperature, application and source of primer, generally, 15-30 nucleotides in length. Shorter primers generally need lower temperature to form stable hybridization duplexes to templates.

The sequences of primers are not required to have perfectly complementary sequence to templates. The sequences of primers may comprise some mismatches, so long as they can be hybridized with templates and serve as primers. Therefore, the primers of this invention are not required to have perfectly complementary sequence to the BLT2 gene as templates; it is sufficient that they have complementarity to the extent that they anneals specifically to the nucleotide sequence of the BLT2 gene for acting as a point of initiation of synthesis. The primer design may be conveniently performed with referring to the BLT2 gDNA or cDNA sequences, preferably, cDNA sequence. For instance, the primer design may be carried out using computer programs for primer design (e.g., PRIMER 3 program). Exemplified primers of this invention is set forth in SEQ ID NO: 9 (sense primer) and SEQ ID NO: 10 (antisense primer).

According to a preferred embodiment, the diagnosis or detection kit for asthma comprising probes is in the form of microarray, more preferably DNA or cDNA microarray, most preferably cDNA microarray.

In microarray, the present probes serve as hybridizable array elements and are immobilized on substrates. A preferable substrate includes suitable solid or semi-solid supporters, such as membrane, filter, chip, slide, wafer, fiber, magnetic or nonmagnetic bead, gel, tubing, plate, macromolecule, microparticle and capillary tube. The hybridizable array elements are arranged and immobilized on the substrate. Such immobilization occurs through chemical binding or covalent binding such as UV. In an embodiment of this invention, the hybridizable array elements are bound to a glass surface modified to contain epoxi compound or aldehyde group or to a polylysin-coated surface. Further, the hybridizable array elements are bound to a substrate through linkers (e.g. ethylene glycol oligomer and diamine).

DNAs to be examined with a microarray of this invention may be labeled, and hybridized with array elements on microarray. Various hybridization conditions are applicable, and for the detection and analysis of the extent of hybridization, various methods are available depending on labels used.

The present method for diagnosing rheumatoid arthritis may be carried out in accordance with hybridization. For such analysis, probes, which have a complementary sequence to the nucleotide sequence of the BLT2 gene, are used.

Using probes hybridizable with the BLT2 gene or cDNA, preferably cDNA, asthma is diagnosed or detected by hybridization-based assay. According to a preferred embodiment, some modifications in the probes of this invention can be made unless the modifications abolish the advantages of the probes. Such modifications, i.e., labels linking to the probes generate a signal to detect hybridization. Suitable labels include fluorophores (e.g., fluorescein), phycoerythrin, rhodamine, lissamine, Cy3 and Cy5 (Pharmacia), chromophores, chemiluminescers, magnetic particles, radioisotopes (e.g., P³² and S³⁵), mass labels, electron dense particles, enzymes (e.g., alkaline phosphatase and horseradish peroxidase), cofactors, substrates for enzymes, heavy metals (e.g., gold), and haptens having specific binding partners, e.g., an antibody, streptavidin, biotin, digoxigenin and chelating group, but not limited to. Labeling is performed according to various methods known in the art, such as nick translation, random priming (Multiprime DNA labeling systems booklet, “Amersham” (1989)) and kination (Maxam & Gilbert, Methods in Enzymology, 65:499 (1986)). The labels generate signal detectable by fluorescence, radioactivity, measurement of color development, mass measurement, X-ray diffraction or absorption, magnetic force, enzymatic activity, mass analysis, binding affinity, high frequency hybridization or nanocrystal.

The nucleic acid sample (preferably, cDNA) to be analyzed may be prepared using mRNA from various biosamples. The biosample is preferably a cell from airway epithelium. Instead of probes, cDNA may be labeled for hybridization-based analysis.

Probes are hybridized with cDNA molecules under stringent conditions for detecting asthma. Suitable hybridization conditions may be routinely determined by optimization procedures. Conditions such as temperature, concentration of components, hybridization and washing times, buffer components, and their pH and ionic strength may be varied depending on various factors, including the length and GC content of probes and target nucleotide sequence. The detailed conditions for hybridization can be found in Joseph Sambrook, et al., Molecular Cloning, A Laboratory Manual, Cold Spring Harbor Laboratory Press, Cold Spring Harbor, N.Y. (2001); and M. L. M. Anderson, Nucleic Acid Hybridization, Springer-Verlag New York Inc. N.Y. (1999). For example, the high stringent condition includes hybridization in 0.5 M NaHPO₄, 7% SDS (sodium dodecyl sulfate) and 1 mM EDTA at 65° C. and washing in 0.1×SSC (standard saline citrate)/0.1% SDS at 68□. Also, the high stringent condition includes washing in 6×SSC/0.05% sodium pyrophosphate at 48° C. The low stringent condition includes e.g., washing in 0.2×SSC/0.1% SDS at 42° C.

Following hybridization reactions, a hybridization signal indicative of the occurrence of hybridization is then measured. The hybridization signal may be analyzed by a variety of methods depending on labels. For example, where probes are labeled with enzymes, the occurrence of hybridization may be detected by reacting substrates for enzymes with hybridization resultants. The enzyme/substrate pair useful in this invention includes, but not limited to, a pair of peroxidase (e.g., horseradish peroxidase) and chloronaphthol, aminoethylcarbazol, diaminobenzidine, D-luciferin, lucigenin (bis-N-methylacridinium nitrate), resorufin benzyl ether, luminol, Amplex Red reagent (10-acetyl-3,7-dihydroxyphenoxazine), HYR (p-phenylenediamine-HCl and pyrocatechol), TMB (3,3,5,5-tetramethylbenzidine), ABTS (2,2-Azine-di[3-ethylbenzthiazoline sulfonate]), o-phenylenediamine (OPD) or naphthol/pyronine; a pair of alkaline phosphatase and bromochloroindolylphosphate (BCIP), nitro blue tetrazolium (NBT), naphthol-AS-B1-phosphate or ECF substrate; and a pair of glucosidase and t-NBT (nitroblue tetrazolium) or m-PMS (phenazine methosulfate). Where probes are labeled with gold particles, the occurrence of hybridization may be detected by silver staining method using silver nitrate.

In these connections, where the present method for diagnosing asthma is carried out by hybridization, it comprises the steps of (i) contacting a nucleic acid sample to a probe having a nucleotide sequence complementary to the nucleotide sequence of the BLT2 gene; and (ii) detecting the occurrence of hybridization.

The signal intensity from hybridization is indicative of asthma. When the hybridization signal to BLT2 cDNA from a sample to be diagnosed is measured to be stronger than normal samples, the sample can be determined to have asthma.

According to a preferred embodiment, the primers of this invention are used for amplification reactions.

The term used herein “amplification reactions” refers to reactions for amplifying nucleic acid molecules. A multitude of amplification reactions have been suggested in the art, including polymerase chain reaction (hereinafter referred to as PCR) (U.S. Pat. Nos. 4,683,195, 4,683,202, and 4,800,159), reverse transcription-polymerase chain reaction (hereinafter referred to as RT-PCR) (Sambrook, J. et al., Molecular Cloning. A Laboratory Manual, 3rd ed. Cold Spring Harbor Press (2001)), the methods of Miller, H. I. (WO 89/06700) and Davey, C. et al. (EP 329,822), ligase chain reaction (LCR)(17, 18), Gap-LCR (WO 90/01069), repair chain reaction (EP 439,182), transcription-mediated amplification (TMA)(19) (WO 88/10315), self sustained sequence replication (WO 90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909 and 5,861,245), nucleic acid sequence based amplification (NASBA) (U.S. Pat. Nos. 5,130,238, 5,409,818, 5,554,517, and 6,063,603), strand displacement amplification and loop-mediated isothermal amplification (LAMP), but not limited to. Other amplification methods that may be used are described in, U.S. Pat. Nos. 5,242,794, 5,494,810, 4,988,617 and in U.S. Ser. No. 09/854,317.

According to the most preferred embodiment, the amplification reaction is carried out in accordance with PCR (polymerase chain reaction) which is disclosed in U.S. Pat. Nos. 4,683,195, 4,683,202, and 4,800,159.

PCR is one of the most predominant processes for nucleic acid amplification and a number of its variations and applications have been developed. For example, for improving PCR specificity or sensitivity, touchdown PCR(24), hot start PCR(25, 26), nested PCR(2) and booster PCR(27) have been developed with modifying traditional PCR procedures. In addition, real-time PCR, differential display PCR (DD-PCR), rapid amplification of cDNA ends (RACE), multiplex PCR, inverse polymerase chain reaction (IPCR), vectorette PCR, thermal asymmetric interlaced PCR (TAIL-PCR) and multiplex PCR have been suggested for certain applications. The details of PCR can be found in McPherson, M. J., and Moller, S. G. PCR. BIOS Scientific Publishers, Springer-Verlag New York Berlin Heidelberg, N.Y. (2000), the teachings of which are incorporated herein by reference in its entity.

Where the present method for diagnosing asthma is carried out using primers, the nucleic acid amplification is executed for analyzing the expression level of the BLT2 gene. Because the present invention is intended to assess the expression level of the BLT2 gene, the level of the BLT2 mRNA in samples is analyzed.

Therefore, the present invention performs nucleic acid amplifications using mRNA molecules in samples as templates and primers to be annealed to mRNA or cDNA.

For obtaining mRNA molecules, total RNA is isolated from samples. The isolation of total RNA may be performed by various methods (Sambrook, J. et al., Molecular Cloning. A Laboratory Manual, 3rd ed. Cold Spring Harbor Press (2001); Tesniere, C. et al., Plant Mol. Biol. Rep., 9:242 (1991); Ausubel, F. M. et al., Current Protocols in Molecular Biology, John Willey & Sons (1987); and Chomczynski, P. et al., Anal. Biochem. 162:156 (1987)). For example, total RNA in cells may be isolated using Trizol. Afterwards, cDNA molecules are synthesized using mRNA molecules isolated and then amplified. Since total RNA molecules used in the present invention are isolated from human samples, mRNA molecules have poly-A tails and converted to cDNA by use of dT primer and reverse transcriptase (PNAS USA, 85:8998 (1988); Libert F, et al., Science, 244:569 (1989); and Sambrook, J. et al., Molecular Cloning. A Laboratory Manual, 3rd ed. Cold Spring Harbor Press (2001)). cDNA molecules synthesized are then amplified by amplification reactions.

The primers used for the present invention is hybridized or annealed to a region on template so that double-stranded structure is formed. Conditions of nucleic acid hybridization suitable for forming such double stranded structures are described by Joseph Sambrook, et al., Molecular Cloning, A Laboratory Manual, Cold Spring Harbor Laboratory Press, Cold Spring Harbor, N.Y. (2001) and Haymes, B. D., et al., Nucleic Acid Hybridization, A Practical Approach, IRL Press, Washington, D.C. (1985).

A variety of DNA polymerases can be used in the amplification step of the present methods, which includes “Klenow” fragment of E. coli DNA polymerase 1, a thermostable DNA polymerase and bacteriophage T7 DNA polymerase. Preferably, the polymerase is a thermostable DNA polymerase such as may be obtained from a variety of bacterial species, including Thermus aquaticus (Taq), Thermus thermophilus (Tth), Thermus filiformis, Thermis flavus, Thermococcus literalis, and Pyrococcus furiosus (Pfu).

When a polymerization reaction is being conducted, it is preferable to provide the components required for such reaction in excess in the reaction vessel. Excess in reference to components of the amplification reaction refers to an amount of each component such that the ability to achieve the desired amplification is not substantially limited by the concentration of that component. It is desirable to provide to the reaction mixture an amount of required cofactors such as Mg²⁺, and dATP, dCTP, dGTP and dTTP in sufficient quantity to support the degree of amplification desired. All of the enzymes used in this amplification reaction may be active under the same reaction conditions. Indeed, buffers exist in which all enzymes are near their optimal reaction conditions. Therefore, the amplification process of the present invention can be done in a single reaction volume without any change of conditions such as addition of reactants.

Annealing or hybridization in the present method is performed under stringent conditions that allow for specific binding between the primer and the template nucleic acid. Such stringent conditions for annealing will be sequence-dependent and varied depending on environmental parameters.

The amplified BLT2 cDNA molecules are then analyzed to assess the expression level of the BLT2 gene. For example, the amplified products are resolved by a gel electrophoresis and the bands generated are analyzed to assess the expression level of the BLT2 gene. When the expression level of the BLT2 gene from a sample to be diagnosed is measured to be higher than normal samples, the sample can be determined to have asthma.

In these connections, where the present method for diagnosing asthma is carried out by amplification, it comprises the steps of (i) amplifying a nucleic acid sample by use of a primer having a nucleotide sequence complementary to the nucleotide sequence of the BLT2 gene; and (ii) analyzing the amplified products to determine the expression level of the BLT2 gene.

In a preferred embodiment, the kit may comprise a pair of primers having a forward sequence of SEQ ID NO: 9 and a reverse sequence of SEQ ID NO: 10. This primer set can detect both of the long form and short form BLT2.

In a preferred embodiment, the kit may comprise a pair of primers having a forward sequence of SEQ ID NO: 11 and a reverse sequence of SEQ ID NO: 12. This primer set can detect only long form of BLT2 because the primer recognizes the front part of long form CDS.

According to another aspect of the present invention, there is provided a kit for detecting asthma, which comprises an antibody binding specifically to BLT2 protein. The diagnosing kit for asthma may be constructed by incorporating an antibody binding specifically to the BLT2 protein.

The antibody against the BLT2 protein used in this invention may polyclonal or monoclonal, preferably monoclonal. The antibody could be prepared according to conventional techniques such as a fusion method (Kohler and Milstein, European Journal of Immunology, 6:511-519 (1976)), a recombinant DNA method (U.S. Pat. No. 4,816,56) or a phage antibody library (Clackson et al, Nature, 352:624-628 (1991) and Marks et al, J. Mol. Biol., 222:58, 1-597 (1991)). The general procedures for antibody production are described in Harlow, E. and Lane, D., Antibodies: A Laboratory Manual, Cold Spring Harbor Press, New York, 1988; Zola, H., Monoclonal Antibodies: A Manual of Techniques, CRC Press, Inc., Boca Raton, Fla., 1984; and Coligan, CURRENT PROTOCOLS IN IMMUNOLOGY, Wiley/Greene, NY, 1991, which are incorporated herein by references. For example, the preparation of hybridoma cell lines for monoclonal antibody production is done by fusion of an immortal cell line and the antibody producing lymphocytes. This can be done by techniques well known in the art. Polyclonal antibodies may be prepared by injection of the BLT2 protein antigen to suitable animal, collecting antiserum containing antibodies from the animal, and isolating specific antibodies by any of the known affinity techniques.

Where the diagnosing method of this invention is performed using antibodies to the BLT2 protein, it could be carried out according to conventional immunoassay procedures for detecting asthma.

Such immunoassay may be executed by quantitative or qualitative immunoassay protocols, including radioimmunoassay, radioimmuno-precipitation, enzyme-linked immunosorbent assay (ELISA), capture-ELISA, inhibition or competition assay, sandwich assay, flow cytometry, immunofluorescence assay and immuoaffinity assay, but not limited to. The immunoassay and immuostaining procedures can be found in Enzyme Immunoassay, E. T. Maggio, ed., CRC Press, Boca Raton, Fla., 1980; Gaastra, W., Enzyme-linked immunosorbent assay (ELISA), in Methods in Molecular Biology, Vol. 1, Walker, J. M. ed., Humana Press, NJ, 1984; and Ed Harlow and David Lane, Using Antibodies: A Laboratory Manual, Cold Spring Harbor Press, 1999, which are incorporated herein by references.

For example, according to the radioimmunoassay method, the radioisotope (e.g., C¹⁴, I¹²⁵, P³² and S³⁵) labeled antibody may be used to detect the BLT2 protein.

In addition, according to the ELISA method, the example of the present method may comprise the steps of: (i) coating a surface of solid substrates with cell lysate to be analyzed; (ii) incubating the coated cell lysate with a primary antibody to the BLT2 protein; (iii) incubating the resultant with a secondary antibody conjugated with an enzyme; and (iv) measuring the activity of the enzyme.

The solid substrate useful in this invention includes carbohydrate polymer (e.g., polystyrene and polypropylene), glass, metal and gel, most preferably microtiter plates.

The enzyme conjugated with the secondary antibody is that catalyzing colorimetric, fluorometric, luminescence or infra-red reactions, e.g., including alkaline phosphatase, β-galactosidase, luciferase, Cytochrome P₄₅₀ and horseradish peroxidase. Where using alkaline phosphatase, bromochloroindolylphosphate (BCIP), nitro blue tetrazolium (NBT) or ECF may be used as a substrate for color-developing reactions; in the case of using horseradish peroxidase, chloronaphthol, aminoethylcarbazol, diaminobenzidine, D-luciferin, lucigenin (bis-N-methylacridinium nitrate), resorufin benzyl ether, luminol, Amplex Red reagent (10-acetyl-3,7-dihydroxyphenoxazine), HYR (p-phenylenediamine-HCl and pyrocatechol), TMB (3,3,5,5-tetramethylbenzidine), ABTS (2,2-Azine-di[3-ethylbenzthiazoline sulfonate]), o-phenylenediamine (OPD) or naphthol/pyronine may be used as a substrate; and in the case of using glucose oxidase, t-NBT (nitroblue tetrazolium) or m-PMS (phenazine methosulfate) may be used as a substrate.

Where the present method is performed in accordance with the capture-ELISA method, the specific example of the present method may comprise the steps of: (i) coating a surface of a solid substrate with a capturing antibody capable of binding specifically to the BLT2 protein; (ii) incubating the capturing antibody with a cell sample to be analyzed; (iii) incubating the resultant of step (ii) with a detecting antibody which is capable of binding specifically to the BLT2 protein and conjugated with a label generating a detectable signal; and (iv) detecting the signal generated from the label conjugated with the detecting antibody.

The detecting antibody has a label generating a detectable signal. The label includes, but not limited to, a chemical (e.g., biotin), an enzymatic (e.g., alkaline phosphatase, horseradish peroxidase, β-galactosidase and Cytochrome P₄₅₀), a radioactive (e.g., C¹⁴, I¹²⁵, P³² and S³⁵), a fluorescent (e.g., fluorescein), a luminescent, a chemiluminescent and a FRET (fluorescence resonance energy transfer) label. Various labels and methods for labeling antibodies are well known in the art (Ed Harlow and David Lane, Using Antibodies, A Laboratory Manual, Cold Spring Harbor Laboratory Press, 1999).

The detection of the signal generated from the label conjugated with the detecting antibody can be carried out by various processes well known in the art. The detection of the signal enables to analyze the BLT2 protein in a quantitative or qualitative manner. Where biotin and luciferase are used as labels, the signal detection may be achieved by use of streptavidin and luciferin, respectively.

The measurement of signal intensities generated from the immunoassay described above is indicative of asthma. When the signal to the BLT2 protein in a biosample to be diagnosed is measured to be higher than normal samples, the biosample can be determined to have asthma.

The kit of the present invention may optionally include other reagents along with primers, probes or antibodies described above. For instance, where the present kit may be used for nucleic acid amplification, it may optionally include the reagents required for performing PCR reactions such as buffers, DNA polymerase (thermostable DNA polymerase obtained from Thermus aquaticus (Taq), Thermus thermophilus (Tth), Thermus filiformis, Thermis flavus, Thermococcus literalis, and Pyrococcus furiosus (Pfu)), DNA polymerase cofactors, and deoxyribonucleotide-5-triphosphates. The kits, typically, are adapted to contain in separate packaging or compartments the constituents afore-described.

The kits for detecting or diagnosing asthma permit to determine the development, aggravation and alleviation of asthma. In this regard, the term used herein “detecting or diagnosing” with reference to disease means not only the determination of the existence of disease but also the development, aggravation and alleviation of disease.

The pharmaceutically acceptable carrier contained in the pharmaceutical composition of the present invention, which is commonly used in pharmaceutical formulations, but is not limited to, includes lactose, dextrose, sucrose, sorbitol, mannitol, starch, rubber arable, potassium phosphate, arginate, gelatin, potassium silicate, microcrystalline cellulose, polyvinylpyrrolidone, cellulose, water, syrups, methylcellulose, methylhydroxy benzoate, propylhydroxy benzoate, talc, magnesium stearate, and mineral oils. The pharmaceutical composition according to the present invention may further include a lubricant, a humectant, a sweetener, a flavoring agent, an emulsifier, a suspending agent, and a preservative. Details of suitable pharmaceutically acceptable carriers and formulations can be found in Remington's Pharmaceutical Sciences (19th ed., 1995), which is incorporated herein by reference.

According to another aspect of the present invention, there is provided a kit for detecting asthma, which comprises a primer or probe having a base sequence complementary to the base sequence of Rac gene. Therefore, any methodology or kit developed based on the information that Rac overexpression is detected in the lung airway of a patient with asthma may be included in the present invention.

According to another aspect of the present invention, there is provided a kit for detecting asthma, which comprises an antibody binding specifically to Rac protein. The diagnosing kit for asthma may be constructed by incorporating an antibody binding specifically to the Rac protein.

A pharmaceutical composition of this invention may be administered orally or parenterally (e.g., intravenous injection, subcutaneous injection, intramuscular injection and local injection).

The term “therapeutically effective amount” as used herein means an amount of the substance that is capable of producing a medically desirable result in a treated subject. The correct dosage of the pharmaceutical compositions of this invention will be varied according to the particular formulation, the mode of application, age, body weight and sex of the patient, diet, time of administration, condition of the patient, drug combinations, reaction sensitivities and severity of the disease. According to a preferred embodiment of this invention, a daily suitable dosage unit for human host ranges from 0.001-100 mg/kg (body weight).

According to the conventional techniques known to those skilled in the art, the pharmaceutical compositions of this invention can be formulated with pharmaceutical acceptable carrier and/or vehicle as described above, finally providing several forms including a unit dosage form. Non-limiting examples of the formulations include, but not limited to, a solution, a suspension or an emulsion, an extract, an elixir, a powder, a granule, a tablet, a capsule, emplastra, a liniment, a lotion and an ointment.

Asthma is a chronic inflammatory disease of the airway characterized by eosinophil infiltration, mucus hypersecretion and AHR. Although there have been many studies of the role of BLT1 in asthma, the role of BLT2 has not yet been defined. By employing a pharmacological BLT2 antagonist and an antisense nucleotide sequence that blocked endogenous BLT2 expression, we demonstrated that BLT2 plays a critical role in the development of AHR and airway inflammation. We also presented evidence that BLT2 mediates the asthmatic response by stimulating ROS generation and subsequent NF-κB activation. Ours is the first report that BLT2 is induced by allergenic stimuli and that blockade of BLT2 mitigates the asthmatic response.

A number of earlier studies have shown that the ‘LTB4-BLT1’ pathway plays a central role in the early chemoattraction of granulocytes such as eosinophils to inflamed tissues, thereby acting as a local inflammatory mediator (3, 35). The LTB4-BLT1 pathway is also considered pivotal for the allergen-mediated recruitment of effector CD4+ and CD8+ T lymphocytes into airways, thereby controlling the immunological response, as well (10-12, 36). LTB4-BLT1 is therefore believed to contribute to the development of asthma through recruitment of granulocytes and effector CD4+ and CD8+ T lymphocytes. In contrast to BLT1, BLT2 has a low affinity for LTB4, and no clear physiological function has yet been identified for it. To our surprise, BLT2 was dramatically upregulated during OVA-induced allergic pulmonary inflammation in our asthma model. Unlike BLT1, which is mainly expressed in myeloid leukocytes and specific classes of T cells, BLT2 was induced in the airway epithelium and in parts of the endothelium (FIG. 1C), where LTB4 is abundantly generated in response to allergen challenge. After aerosol challenge with OVA, airway mast cells and alveolar macrophages are activated. These cells are thought to be the major source of LTB4 in the airways at early times following allergen challenge. Although LTB4 is believed to attract eosinophils, neutrophils and differentiated T cells to airways via the BLT1 they express, LTB4 may also interact with BLT2 induced in the airway epithelium in the local microenvironment and stimulate intracellular signaling leading to upregulation of VCAM-1 and other proinflammatory proteins. The induced VCAM-1 could in turn enhance trafficking of inflammatory leukocytes across the epithelial layer to the airways, thereby contributing to a development of airway inflammation and AHR. In fact, leukocyte emigration into the alveolar compartments is a prominent feature of acute and chronic inflammatory lung injuries such as asthma (37). During this emigration process, airway epithelial cells are probably important not only for retention and activation of leukocytes, but also for regulating their passage into the airways. In any event, the distribution and function of ‘LTB4-BLT2’ during the asthmatic response appear to be unique and distinguishable from that of LTB4-BLT1. BLT2 clearly plays only a minimal role in T cell recruitment; consequently, T cell recruitment to airways remained intact after administration of antisense BLT2 (FIG. 8), while AHR and airway inflammation were attenuated. This means the mechanism by which BLT2 inhibition suppresses AHR and airway inflammation is independent of T cell recruitment. On the other hand, no expression of BLT2 was detected in CD4+ T cells and CD8+ TEFF cells, though they strongly express BLT1 (10, 38).

We suggest that ROS is a major downstream component of the LTB4-BLT2 pathway mediating AHR and airway inflammation in allergic asthma. Accumulating evidence suggests that ROS and the oxidative stress they cause play crucial roles in the pathogenesis of airway inflammation and AHR (29, 39-41). At later stages, moreover, the inflammatory cells recruited to the asthmatic airways have the capability to produce ROS, and the ROS released by eosinophils and other leukocytes infiltrating the airways cause the tissue injury observed in asthma (42).

That said, in this case we do not believe ROS are acting merely as nonspecific pathogenic mediators of oxidative stress. Instead, we suggest they have a specific signaling function in the pathway leading to upregulation of target genes associated with asthma. In support of this idea, we previously showed that BLT2 activity leads to enhanced ROS generation, which in turn mediates specific intracellular signaling responses (7, 37). Although the molecular basis of ROS-mediated induction of AHR and inflammation remains unknown, recent studies have shown that ROS generation in asthma leads to activation of the redox-sensitive transcription factor NF-κB (32, 43), which is present in most cell types and plays a critical role in immune and inflammatory responses, including asthma (44). For instance, NF-κB activation contributes to the development and maintenance of asthma in the bronchial epithelium (45), and has been observed in airway epithelial cells (46) in which BLT2 mRNA is induced during the OVA-induced allergic response. Consistent with those observations, we found in the present study that NF-κB levels were substantially elevated in extracts of lung tissue from mice with OVA-induced asthma and were specifically suppressed by BLT2 antisense (FIG. 6A). It is known that activation of NF-κB induces a variety of pro-inflammatory genes, including adhesion molecules (e.g., VCAM-1) (33, 34). As expected, expression of VCAM-1 increased following allergen challenge, and BLT2 antisense or a receptor antagonist reduced its expression (FIGS. 6C and 6E), whereas BLT1 antisense had no effect (data not shown).

BLT2−/− knockout mice are difficult to generate because the BLT2 gene resides within the BLT1 locus (47). Disruption of BLT2 therefore interferes with BLT1 expression, making it difficult to interpret the outcome. Therefore, we recently prepared transgenic mice overexpressing BLT2 and detected elevated levels of ROS in their BAL fluid (Cho et al., unpublished observation). In addition, we observed significant induction of AHR, even before OVA challenge, which further supports the proposed mediatory role of BLT2 in the pathogenesis of asthma (Cho et al., unpublished observation).

This is the first report of a relationship between asthma and a BLT2-linked signaling cascade. We suggest that LTB4 exerts its effects through both BLT1- and BLT2-dependent signaling pathways and that the two may cooperate during the development of allergic asthma, although attenuation of either pathway suppressed asthmatic symptoms. A better understanding of the BLT2-linked pathway and possible cross-regulation between the BLT1 and BLT2 pathways should help to clarify their role in LTB4-mediated allergic pathogenesis. Beyond that, our finding that a LTB4-BLT2-ROS pathway is involved in asthma could serve as the basis for the development of new diagnostic tools and treatments for allergic disease.

### BRIEF DESCRIPTION OF THE DRAWINGS

The above object and advantages of the present invention will become more apparent by describing in detail preferred embodiments thereof with reference to the attached drawings in which:

FIG. 1 shows increased expression of BLT2 mRNA in an OVA-induced murine asthma model. A, Levels of LTB4 in BAL fluid measured using an ELISA at the indicated times following OVA challenge. B, Semiquantitative RT-PCR analysis of BLT1 and BLT2. Levels of BLT2 mRNA in the lungs were measured in control (normal), pre-OVA challenged (pre-provocation) and OVA-challenged (OVA provocation) mice. GAPDH was used as quantitative control. C, In situ hybridization of BLT2 mRNA in lung airways. The distributions of BLT2 mRNA in normal (i, ii) and OVA-induced asthmatic mouse lung airways (iii, iv) are shown (arrows). Data are means±SEM (n=4 in each group). Magnification, 100□ (i, ii) or 200□ (iii, iv).

FIG. 2 shows that LY255283 attenuates airway inflammation in asthma. BALB/c mice were intravenously injected with LY255283 (2.5 mg/kg) or vehicle (DMSO) 1 h before 1% OVA challenge. The mice were then killed on day 25 to assess asthmatic phenotypes. A, Semiquantitative RT-PCR analysis of BLT2 mRNA levels in lung tissue. B, Quantitative analyses of BLT2 mRNA levels using real-time PCR. C-E, Histological analysis of lung airways from OVA-challenged mice 48 h after the last 1% OVA challenge. Lungs were excised, fixed, and stained with HE. Mice were sensitized/challenged with PBS (Normal, C), OVA with DMSO pretreatment (OVA/DMSO, D), or OVA with LY255283 (2.5 mg/ml) pretreatment (OVA/LY255283, E). Scale Bars, 50 μm. F, Histological scores. The data are means±SEM (n=5 in each group). *P<0.05 vs. OVA/DMSO.

FIG. 3 shows that antisense BLT2 attenuates airway inflammation in asthma. A, Normal and OVA-challenged mice (C57BL/6) pretreated with sense (ss) BLT2 (1.6 mg/kg), antisense (as) BLT2 (1.6 mg/kg) or buffer (saline) 24 h and 1 h before 10% OVA challenge were sacrificed two days after the last OVA challenge, and their lungs were analyzed for levels of BLT1, BLT2 and control GAPDH mRNAs using semiquantitative RT-PCR analysis. B, Quantitative analyses of BLT2 mRNA levels using real-time PCR. C, Infiltration of eosinophils into BAL. Eosinophils (arrows) in BAL fluid were obtained using cytospin and stained with Diff-Quick. Scale Bars, 50 μm. D, Histological analysis of lung airways from OVA-challenged mice 48 h after OVA challenge. Lungs were excised, fixed, and stained with HE. For this experiment, mice were pretreated with sense BLT2 (1.6 mg/kg), antisense BLT2 (1.6 mg/kg) or buffer (saline) before OVA challenge. Scale Bars, 50 μm. Data are means±SEM (n=5 in each group). *P<0.05 vs. OVA/ssBLT2; **P<0.01 vs. OVA/ssBLT2; ***P<0.001 vs. OVA/ssBLT2.

FIG. 4 shows effect of BLT2 inhibition on AHR. A-B, Effect of LY255283 (A) or antisense BLT2 (B) on AHR in OVA-challenged mice. AHR was measured 24 h after the last 1% OVA challenge, after which mice was placed in a chamber and nebulized with increasing doses of methacholine (6.25 mg/ml ˜50 mg/ml) for 3 min. Data are means±SEM (n=5 in each group). *P<0.05 vs. OVA/DMSO in A or OVA/ssBLT2 in B.

FIG. 5 shows that antisense BLT2 attenuates ROS generation. A, BAL fluid was collected 48 h after 10% OVA challenge. Normal and OVA-challenged mice were pretreated with sense BLT2 (1.6 mg/kg), antisense BLT2 (1.6 mg/kg) or buffer (saline) 24 h and 1 h before 10% provocation and then sacrificed at 48 h after the last OVA challenge. The cells present in the BAL fluid were washed and then immediately observed using a FACSCalibur™. B, Measurement of LTB4 levels in BAL fluid using a specific ELISA. Normal and OVA-challenged mice were pretreated with sense BLT2 (1.6 mg/kg), antisense BLT2 (1.6 mg/kg) or buffer (saline) 24 h and 1 h before 10% provocation and then sacrificed at 48 h after the last OVA challenge. BAL fluid was then collected for LTB4 analysis. Data are means±SEM (n=5 in each group). *P<0.05 vs. OVA/ssBLT2; **P<0.01 vs. OVA/ssBLT2.

FIG. 6 shows that BLT2 inhibition attenuates NF-κB activation and VCAM-1 expression. A, EMSA analysis of NF-κB activation following OVA-challenge. Normal and OVA-challenged mice were pretreated with sense BLT2 (1.6 mg/kg), antisense BLT2 (1.6 mg/kg) or buffer (saline) 24 h and 1 h before provocation and then sacrificed two days after the last OVA challenge. Nuclear extracts were then prepared from the lungs and incubated with labeled NF-κB-binding oligonucleotides. B-C, Lung tissue extracts were prepared from normal mice, OVA-challenged mice (OVA/saline), OVA-challenged mice administered sense BLT2 (1.6 mg/kg) or antisense BLT2 (1.6 mg/kg). Equal amounts of protein were then analyzed by immunoblotting with antibodies against IκB-α (B) and VCAM-1 (C). Tubulin was used as a loading control. D-E, Normal and OVA-challenged mice were pretreated with DMSO or LY255283 (2.5 mg/kg) 1 h before 1% OVA challenge and then sacrificed at 48 h after the last OVA challenge. Lung tissue extracts were then prepared for western blotting. Also shown the relative levels of IκB-α (D) and VCAM-1 (E) obtained using densitometry. The data are means±SEM (n=5 in each group).

FIG. 7 shows increased expression of BLT2 in bronchial biopsy specimens. Biopsy specimens were obtained from healthy subjects (A-C) and subjects with mild (D-E) or moderate (F) bronchial asthma, after which the patterns of BLT2 expression in sections of mucosa were visualized immunohistochemically. Positive signals were colored red using a streptavidin-alkaline phosphatase system, and the cells were counter-stained using hematoxylin. The images shown are representative of experiments with similar results (n=4 for healthy controls and mild bronchial asthma patients; n=5 for moderate bronchial asthma patients). Scale bars, 50 μm. *P<0.05 vs. Normal

FIG. 8 shows recruitment of T lymphocytes into airways. Recruitment of CD4+ (A) and CD8+ (B) T cells into the BAL fluid of mice 12 h after aerosol OVA challenge. BAL fluid was collected 12 h after 10% OVA challenge and washed with PBS. The leukocytes present were stained with FITC-conjugated anti-mouse TCRE□ chain and PE-cy5 anti-mouse CD8a or PE rat anti-mouse CD4 after blocking with anti-FcRγ antibody. Samples were then analyzed by flow cytometry to assess T lymphocyte recruitment. Data are means±SEM (n=6 in each group). *P<0.05 vs. OVA/asBLT2.

FIG. 9 shows effect of antisense Rac oligonucleotide treatment on lung inflammation and NF-κB activation. (A) To check the involvement of Rac in the process of eosinophil infiltration into the lung airway caused by OVA provocation, antisense oligonucleotides (1.25 mg/kg of weight) were injected into the tail vein of the mice 24 hr and 4 hr before provocation. Fourty eight hours after OVA provocation, mice were sacrificed and BAL fluids and lung tissues were obtained. Cells in the BAL fluid were attached to the slide glass and stained with Hemacolour as manufacturer's recommendation. Lung tissues were obtained from normal, buffer, control Rac oligonucleotide (control Rac) or antisense Rac oligonucleotide (asRac) treated mice after OVA provocation. Lung tissues were fixed with 10% formaline, dehydrated and embedded in paraffin. The tissues were cut into 6 μm sections, and stained with Hematoxylin & Eosin. (B) To examine whether OVA provocation induce the NF-κB activity and whether the induced NF-κB is mediated by Rac, electrophoretic mobility shift assay (EMSA) was accompanied using antisense Rac oligonucleotide treated mice. Nuclear extracts were purified from the lung tissues and incubated with 32P-labeled double strand oligonucleotides containing NF-κB binding consensus or mutant sequence at room temperature. DNA-protein complexes were separated by electrophoresis in 6% acrylamide gel under nondenaturing condition, the autoradiography was performed. (C) To check expression level of VCAM-1, which is known to have critical role in the process of eosinophil transendothelial migration from the blood vessel into the lung parenchyma whole cell lysates were obtained from the lung tissues and western blot analysis was carried out.

FIG. 10 shows activation of Rac in the lung tissue by OVA provocation and inhibition of endogenous Rac expression by antisense Rac oligonucleotide treatment. Lung samples were homogenized with micropestle and washed with PBS twice. (A) For membrane protein preparation, cells were suspended in Buffer A and cells were ruptured by passing through 21-G syringe. After ultracentrifugation, protein samples in pelleted membrane fraction were dissolved with buffer A containing 1% Triton X-100. (B) For whole cell lysate preparation, cells from the lung tissues of buffer, control Rac oligonucleotide or antisense Rac oligonucleotide injected mice were suspended with lysis buffer and incubated for 20 min. After centrifugation of the samples, protein quantification was carried out using Bradford reagent. And western blot analysis was performed.

FIG. 11A shows the suppression effect of BLT2 antisense oligonucleotide on BLT2 expression level by RT-PCR. FIG. 11B shows the suppression effect of BLT2 siRNA on BLT2 expression level by Northern blot.

### BEST MODE FOR CARRYING OUT THE INVENTION

Practical and presently preferred embodiments of the present invention are illustrated as shown in the following Examples. However, it will be appreciated that those skilled in the art, on consideration of this disclosure, may make modifications and improvements within the spirit and scope of the present invention.

### [Reagents]

2′,7′-dichlorofluorescein diacetate (DCF-DA) was purchased from Molecular Probes (Eugene, Oreg.). BSA and DMSO were from Sigma-Aldrich (St. Louis, Mo.). Acetyl-methacholine chloride was purchased from Sigma-Aldrich (St. Louis, Mo.). All other chemicals were from standard sources and were of molecular biology grade or higher. All mice were maintained and bred under specific pathogen-free conditions in the Korea University mouse facility, and experiments were conducted within the parameters of our approved protocol by the Animal Research Committee.

### Example 1

### Sensitization and Challenge of Mice

Female BALB/c mice and C57BL/6 mice (7 weeks old; 18-20 g) were obtained from Orientbion Inc. (Seoungnam, Korea). Sensitization and challenge were carried out as described previously with some modification (28). Briefly, female C57BLU6 mice (7 weeks old; 18-20 g) were immunized by intraperitoneal (i.p.) injection of 200 μg ovalbumin (OVA) emulsified in 2.5 mg of adjuvant aluminum hydroperoxide gel (alum) (Pierce, Rockford, Ill.). A second i.p. injection of 20 μg OVA adsorbed onto alum (2.5 mg) was administered 10 days later. After an additional 10 days, mice were exposed to an aerosol of 1% OVA in saline for 30 min daily on 3 consecutive days. On day 25, mice were finally challenged by provocation with 10% OVA aerosol. For inhibition experiments, sense or antisense BLT2 (1.6 mg/kg) was injected intravenously 24 h and then 1 h before the 10% OVA challenge. The mice were then killed on day 27 to assess asthmatic phenotypes. Antisense BLT2 oligonucleotide (5′-GCTCAGTAGTGTCTCATTCC-3′), sense BLT2 oligonucleotide (5′-GGAATGAGACACTACTGAGC-3′).

Alternatively, BALB/c mice were sensitized on day 1 by i.p. injection of 20 μg OVA emulsified in 2.5 mg of alum (Pierce, Rockford, Ill.), followed by an identical booster injection administered on day 14. On days 21, 22 and 23 after initial sensitization, the mice were challenged for 30 min with an aerosol of 1% OVA using an ultrasonic nebulizer. LY255283 (2.5 mg/kg) or vehicle control (DMSO) was administered intravenously 1 h before 1% OVA challenge. Mice were killed on day 25, to assess asthmatic phenotypes.

### Example 2

### Induction of BLT2 mRNA in the OVA-Induced Asthmatic Mouse Lung

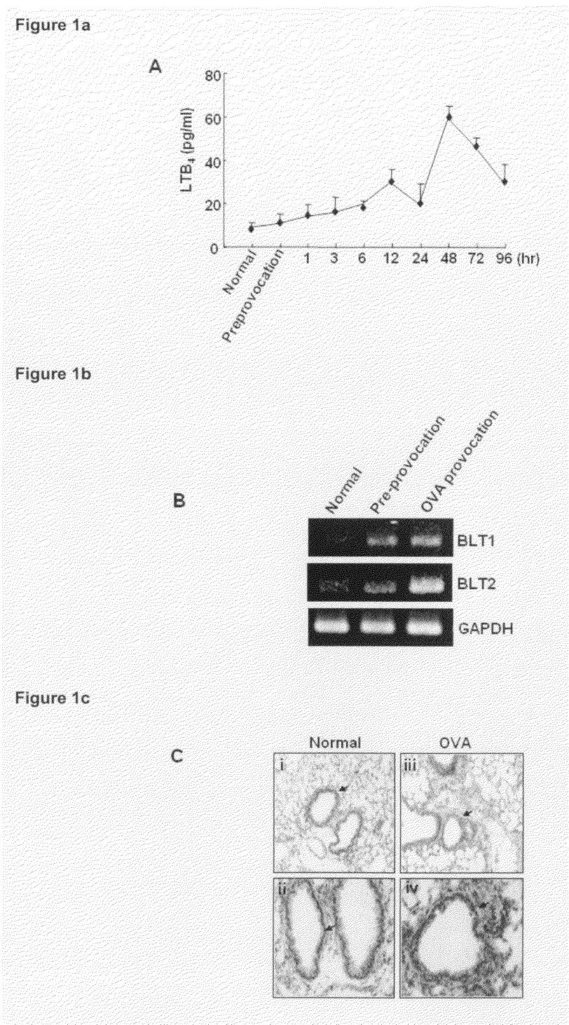

### 1) Quantification of LTB4 (FIG. 1A)

Levels of LTB4 were quantified with the leukotriene B4 enzyme immunoassay (EIA) Biotrak™ system (Amersham Biosciences, UK). Briefly, 200 μl BAL fluid were concentrated by freeze-drying for 12 h and reconstituted in assay buffer. The sensitivity of the assay was 0.3 μg/well, which is equivalent to 6 μg/ml.

### 2) Semiquantitative RT-PCR (FIG. 1B)

Total RNA was extracted from lung samples using Easy-blue RNA extraction reagent (Intron, Korea). The extracted RNA (1 μg) was reverse transcribed for 1 h at 42° C. and amplified by PCR using the following primers: for mouse BLT2,5′-CAGCATGTACGCCAGCGTGC-3′ (sense) and 5′-CGATGGCGCTCACCAGACG-3′ (antisense); and for mouse BLT1,5′-GCATGTCCCTGTCTCTGTTG-3′ (sense) and 5′-CGGGCAAAGGCCTTAGTACG-3′ (antisense). For the semiquantitative analysis of transcripts, we first determined the optimal PCR conditions by linear amplification of glyceraldehyde-3-phosphate dehydrogenase (GAPDH). Thereafter, 28 cycles was used for BLT amplification and was found to be in the linear range. The amplified PCR products were separated by electrophoresis on 1.2% agarose gel and stained with ethidium bromide.

### 3) In Situ Hybridization for BLT2 in Mouse (FIG. 1C)

The cDNA encoding mouse BLT2 was amplified by PCR with the mouse BLT2 primers and confirmed by sequencing. All linearized vectors were transcribed with T7 RNA polymerase and digoxigenin (DIG) RNA labeling mix (Roche, Germany). Embedded mouse lung tissues were deparaffinized with xylene, after which in situ hybridization was carried out using an in situ hybridization detection kit (InnoGenex, CA) according to the manufacturer's protocol.

### Example 3

### Blockade of BLT2 Signaling Using a Pharmacological Antagonist (LY255283) Suppresses Airway Inflammation

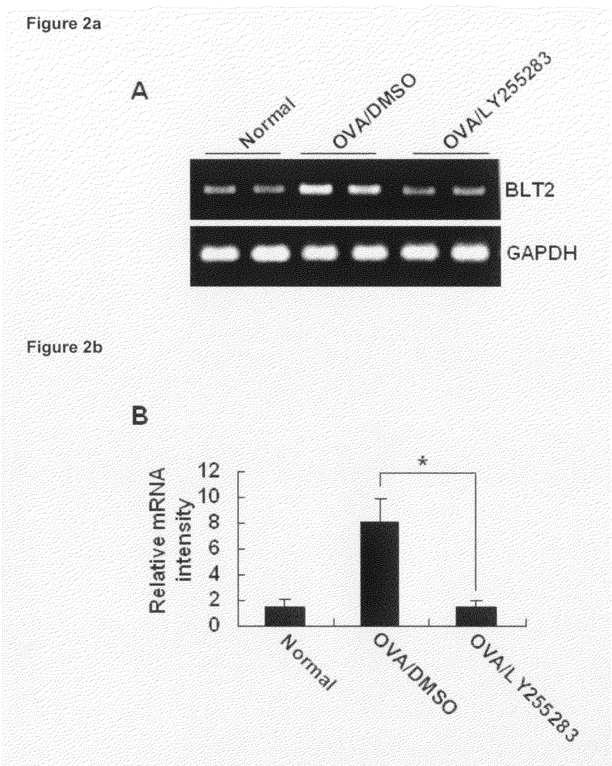

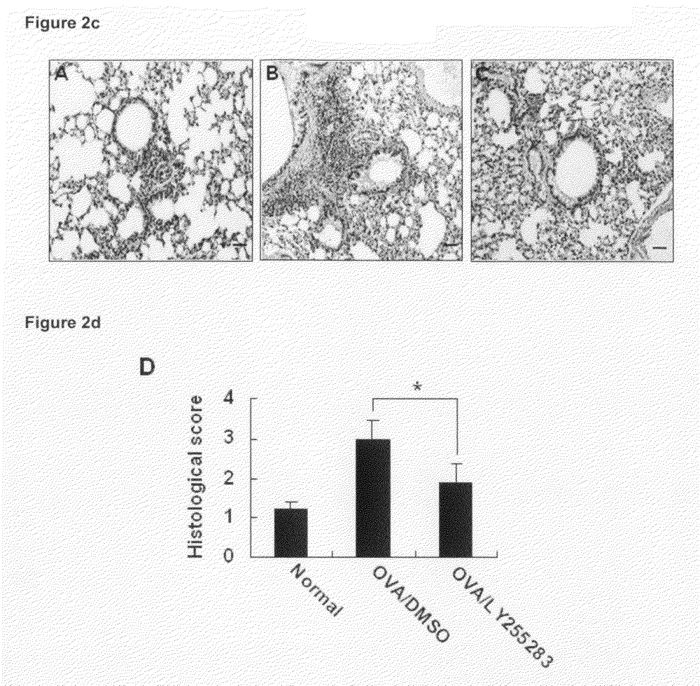

### 1) Semiquantitative RT-PCR (FIG. 2A)

Total RNA was extracted from lung samples using Easy-blue RNA extraction reagent (Intron, Korea). The extracted RNA (1 μg) was reverse transcribed for 1 h at 42° C. and amplified by PCR using the following primers: for mouse BLT2,5′-CAGCATGTACGCCAGCGTGC-3′ (sense) and 5′-CGATGGCGCTCACCAGACG-3′ (antisense); and for mouse BLT1,5′-GCATGTCCCTGTCTCTGTTG-3′ (sense) and 5′-CGGGCAAAGGCCTTAGTACG-3′ (antisense). For the semiquantitative analysis of transcripts, we first determined the optimal PCR conditions by linear amplification of glyceraldehyde-3-phosphate dehydrogenase (GAPDH). Thereafter, 28 cycles was used for BLT amplification and was found to be in the linear range. The amplified PCR products were separated by electrophoresis on 1.2% agarose gel and stained with ethidium bromide.

### 2) Real-Time PCR (FIG. 2B)

For real-time PCR, total RNA were extracted from lung tissue using Easy-blue RNA extraction reagent (Intron, Korea), after which the extracted RNA was reverse transcribed using M-MLV reverse transcriptase (Invitrogen, CA). The PCR reactions were then carried out using LightCycler 480 SYBR Green I Master (Roche, Germany) according to the manufacturer's instructions.

### 3) BAL and Histological Analysis of Lung (FIG. 2C-D)

Inflammatory cells in the BAL fluid were collected by centrifugation (1,000 g for 3 min) and washed once in PBS. Cells were counted using a hemocytometer, and viability was assessed by trypan blue exclusion. In addition, cytospin was carried out for each BAL sample, which was then stained with Diff-Quick (Merck, Dorset, U.K.), enabling differential cell counts to be made. For histological analysis, the lungs of the mice were dissected 48 h after OVA challenge and fixed with 10% formaldehyde in PBS, dehydrated in ethanol-xylene, and embedded in paraffin. Multiple paraffin-embedded 6-μm sections were placed on 0.5% gelatin-coated slides, deparaffinized, and stained with hematoxylin-eosin (HE). Images were acquired using a BX51 microscope (Olympus, Tokyo, Japan) equipped with a DP71 digital camera (Olympus, Tokyo, Japan).

### Example 4

### Blockade of BLT2 Signaling Using a Pharmacological Antisense BLT2 Suppresses Airway Inflammation

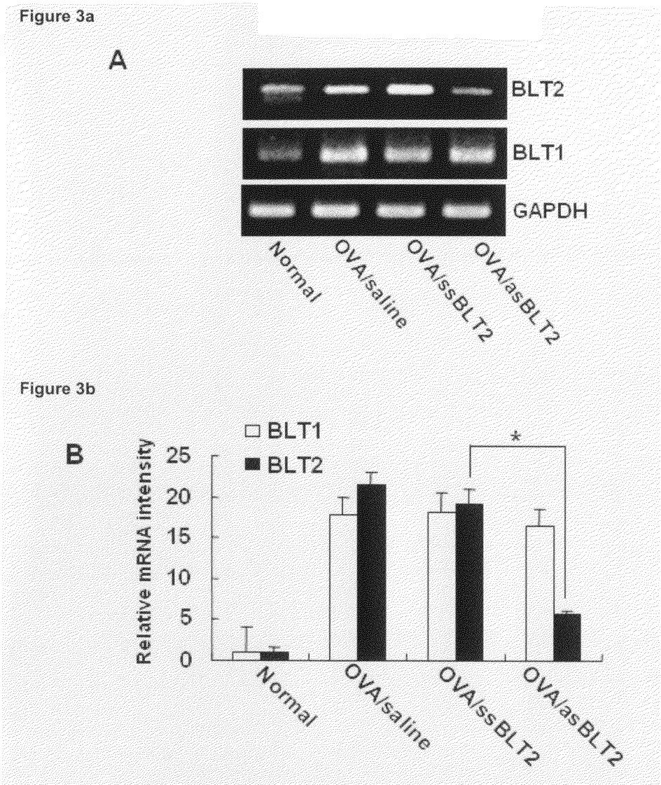

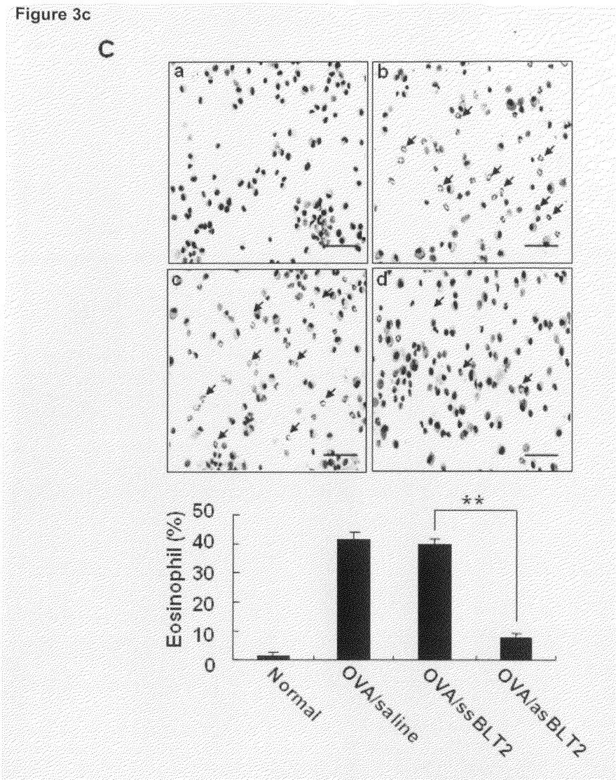

### 1) Semiquantitative RT-PCR (FIG. 3A)

Total RNA was extracted from lung samples using Easy-blue RNA extraction reagent (Intron, Korea). The extracted RNA (1 μg) was reverse transcribed for 1 h at 42° C. and amplified by PCR using the following primers: for mouse BLT2,5′-CAGCATGTACGCCAGCGTGC-3′ (sense) and 5′-CGATGGCGCTCACCAGACG-3′ (antisense); and for mouse BLT1,5′-GCATGTCCCTGTCTCTGTTG-3′ (sense) and 5′-CGGGCAAAGGCCTTAGTACG-3′ (antisense). For the semiquantitative analysis of transcripts, we first determined the optimal PCR conditions by linear amplification of glyceraldehyde-3-phosphate dehydrogenase (GAPDH). Thereafter, 28 cycles was used for BLT amplification and was found to be in the linear range. The amplified PCR products were separated by electrophoresis on 1.2% agarose gel and stained with ethidium bromide.

### 2) Real-Time PCR (FIG. 3B)

For real-time PCR, total RNA were extracted from lung tissue using Easy-blue RNA extraction reagent (Intron, Korea), after which the extracted RNA was reverse transcribed using M-MLV reverse transcriptase (Invitrogen, CA). The PCR reactions were then carried out using LightCycler 480 SYBR Green I Master (Roche, Germany) according to the manufacturer's instructions.

### 3) BAL and Histological Analysis of Lung (FIG. 3C-D)

Inflammatory cells in the BAL fluid were collected by centrifugation (1,000 g for 3 min) and washed once in PBS. Cells were counted using a hemocytometer, and viability was assessed by trypan blue exclusion. In addition, cytospin was carried out for each BAL sample, which was then stained with Diff-Quick (Merck, Dorset, U.K.), enabling differential cell counts to be made. For histological analysis, the lungs of the mice were dissected 48 h after OVA challenge and fixed with 10% formaldehyde in PBS, dehydrated in ethanol-xylene, and embedded in paraffin. Multiple paraffin-embedded 6-μm sections were placed on 0.5% gelatin-coated slides, deparaffinized, and stained with hematoxylin-eosin (HE). Images were acquired using a BX51 microscope (Olympus, Tokyo, Japan) equipped with a DP71 digital camera (Olympus, Tokyo, Japan).

### Example 5

### Effect of BLT2 Inhibition on Airway AHR

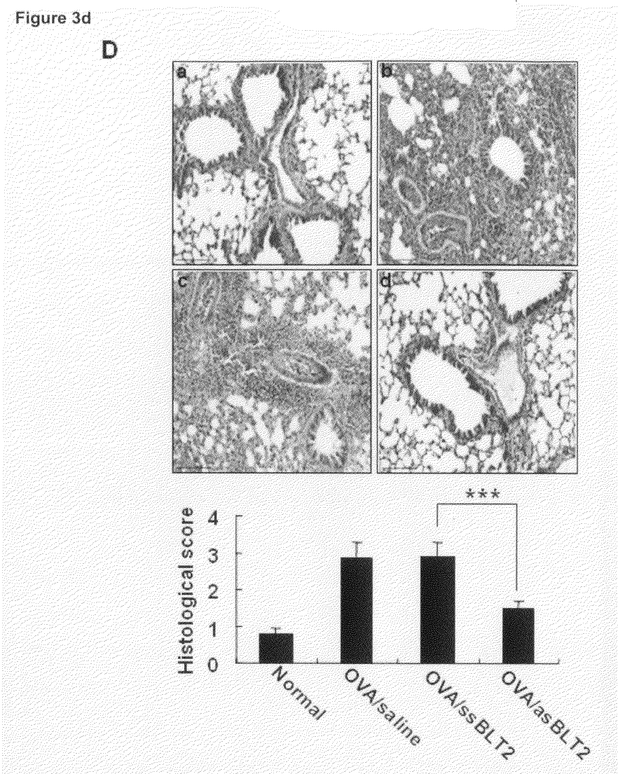

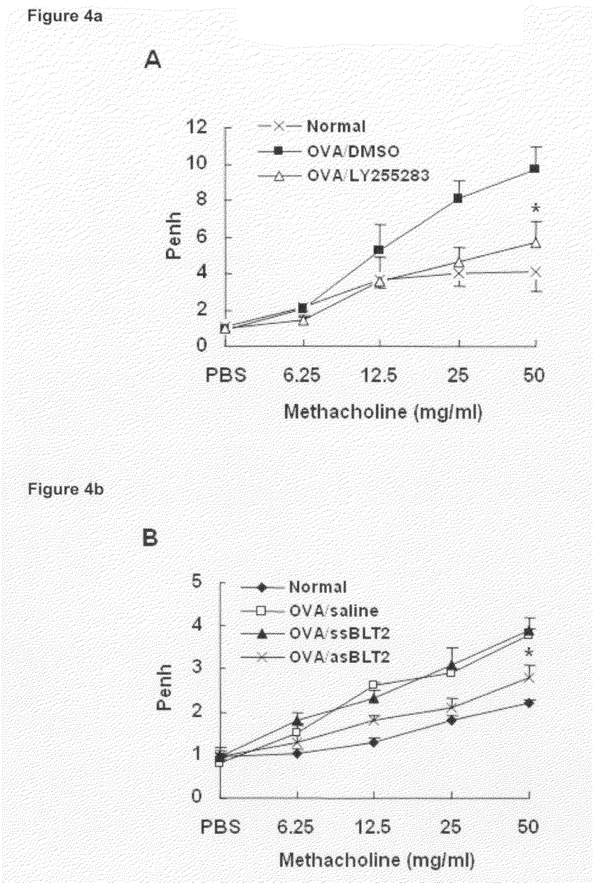

### 1) Determination of AHR in Response to Methacholine (FIG. 4A-B)

Airway AHR was measured in unrestrained, conscious mice 24 h after the final OVA challenge using a whole-body plethysmograph, as previously described (29). Mice were placed in a barometric plethysmographic chamber (All Medicus Co., Seoul, Korea), and baseline readings were taken and averaged for 3 min. Aerosolized methacholine in increasing concentrations (from 6.25 mg/ml-50 mg/ml) was nebulized through an inlet of the main chamber for 3 min. Readings were taken and averaged for 3 min after each nebulization, and enhanced pause (Penh) was determined. Signals were analyzed to derive whole body flow parameters including respiratory rate, tidal volume, inspiratory time (Ti), expiratory time (Te), peak inspiratory flow (PIF), peak expiratory flow (PEF), and relaxation time (RT). These parameters were used to calculate enhanced pause (Penh), a unitless parameter that is used as a measure of airway responsiveness to methacholine. Penh reflects changes in pulmonary resistance during bronchoconstriction according to the following equation: Penh=[(Te−RT)÷RT]×(PEF÷PIF). Baseline Penh measurements for each animal were recorded for 3 min and averaged. Results are expressed as the percentage increase of Penh following challenge with each concentration of methacholine, where the baseline Penh (after saline challenge) is expressed as 100%. Penh values averaged for 3 min after each nebulization were evaluated.

### Example 6

### Attenuation of ROS Generation by BLT2 Inhibition

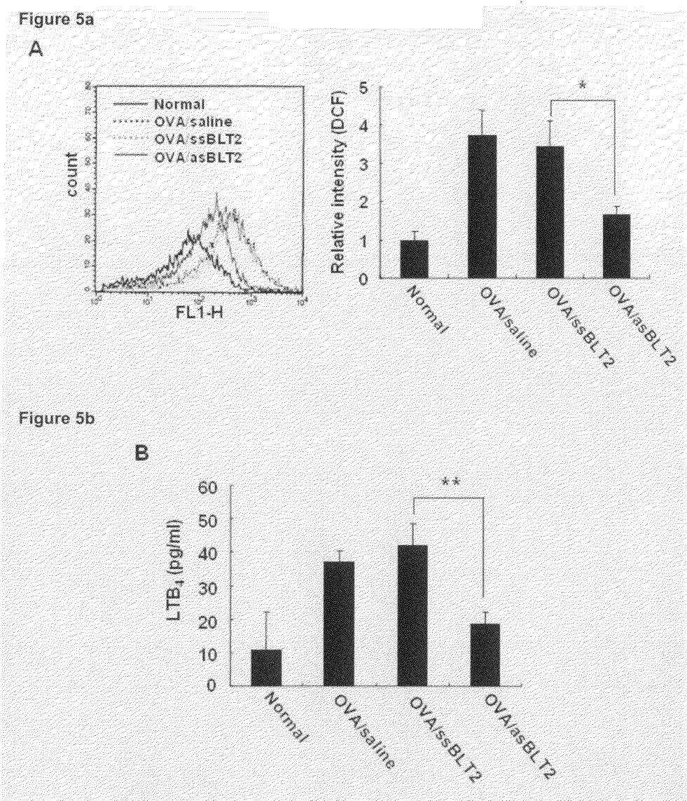

### 1) Measurement of ROS Levels in BAL Fluid (FIG. 5A)

ROS levels in BAL fluids were measured as a function of DCF fluorescence as described previously(30). Briefly, cells in the BAL fluid were collected by centrifugation (1,000 g for 3 min) and the pelleted cells were washed with PBS and incubated for 10 min with the H2O2-sensitive fluorophore 2′,7′-dichorofluorescein diacetate (DCF-DA, 10 μM) (Molecular Probes, Eugene, Oreg.), which, when taken up, fluorescently labels intracellular H2O2 with DCF. Following washing, the cells were immediately observed using a FACS Calibur™ (Becton Dickinson, Franklin Lakes, N.J.). DCF fluorescence was excited at 488 nm and the evoked emission was filtered with a 515-nm long-pass filter.

### 2) Quantification of LTB4 (FIG. 5B)

Levels of LTB4 were quantified with the leukotriene B4 enzyme immunoassay (EIA) Biotrak™ system (Amersham Biosciences, UK). In brief, 200 μl BAL fluid was concentrated by freeze-drying for 12 h and reconstituted in assay buffer. The assay was calibrated with standard LTB4 ranging from 0.31 to 40 μg/well. Samples of BAL fluid and standard LTB4 in 96-well plates were incubated with antiserum for 2 h, followed by LTB4 peroxidase conjugate for 1 h at room temperature. To remove unbound ligand, the wells were aspirated and washed 4 times with buffer. Substrate (tetramethylbenzidine) was then added, and the reaction was stopped by adding an acid solution and the color read at 450 nm in a spectrophotometer. The sensitivity of the assay was 0.3 μg/well, which is equivalent to 6 μg/ml. Statistical significance of differences between groups was assessed by analysis of variance, and P<0.05 was considered significant.

### Example 7

### Attenuation of NF-κB Activation by BLT2 Inhibition

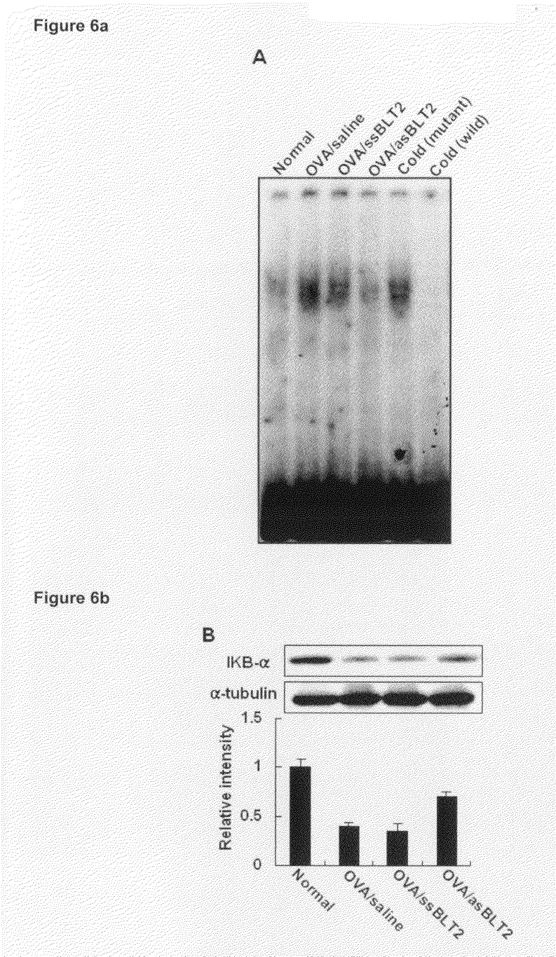

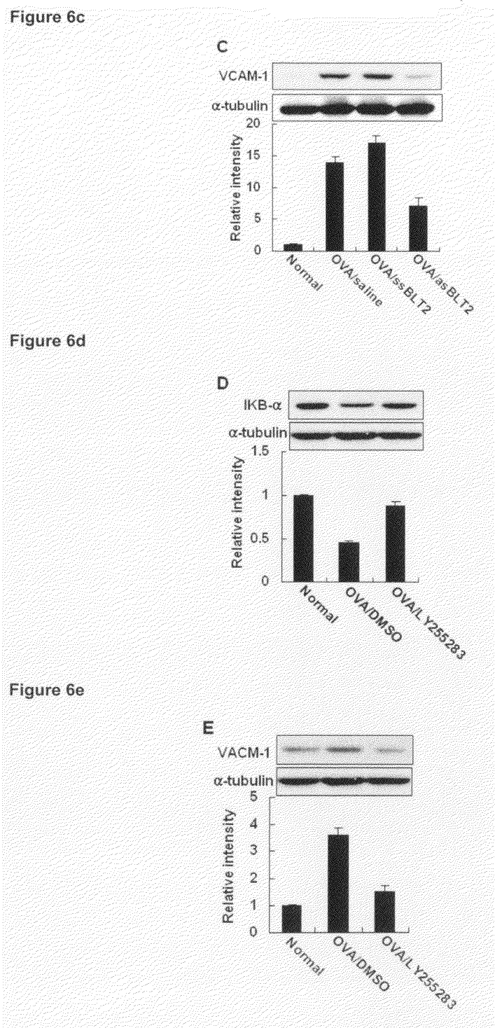

### 1) Electrophoretic Mobility Shift Assay (EMSA) (FIG. 6 A)

A double-strand oligonucleotide corresponding to the consensus NF-κB binding motif and a mutant sequence were purchased from Santa Cruz Biotechnology Inc. (Santa Cruz, Calif.) and labeled with γ-32P-ATP using T4 polynucleotide kinase (Roche, Germany). Labeled oligonucleotide was then separated from free γ-32P-ATP on ProbeQuart™ G-50 microcolumns (Amersham Pharmacia Biotech, Ltd., UK) according to the manufacturer's protocol. Labeled oligonucleotide, 10 μg of nuclear extract and EMSA buffer were incubated for 1 h at room temperature in a final volume of 20 μl, after which the reaction mixture was subjected to electrophoresis.

### 2) SDS-PAGE and Immunoblot Analysis of Lung Cell Lysates (FIG. 6B-E)

Lung samples were prepared as described for preparation of total protein. The cell lysates were centrifuged at 13,000 g for 10 min and the supernatants subjected to SDS-PAGE on 10% acrylamide gels, followed by transfer to polyvinylidene difluoride (PVDF) membranes with a Novex wet transfer system (for 2 h at 100 V). The membranes were blocked for 1 h in Tris-buffered saline (TBS) containing 0.05% (v/v) Tween 20 plus 5% (w/v) nonfat dry skim milk, and then incubated for 2 h with the primary antibody in TBS containing 0.05% (v/v) Tween 20 plus 3% (w/v) BSA, followed for 1 h with horseradish peroxidase (HRP)-conjugated secondary antibody before development by enhanced chemiluminescence (ECL) (Amersham Pharmacia Biotech, Ltd., UK).

### Example 8

### Enhanced Expression of BLT2 in Samples from Clinically Asthmatic Subjects

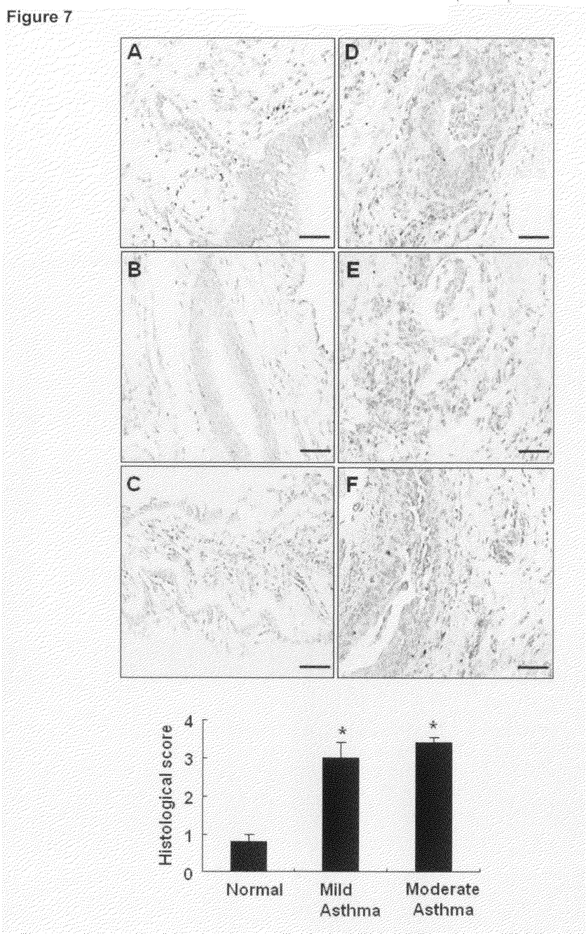

### 1) Bronchoscopy (FIG. 7A-F)

Bronchial biopsy specimens were obtained from 4 nonasthmatic controls (normal), 4 mild bronchial asthma patients and 5 moderate bronchial asthma patients. The patients studied were recruited from the outpatient clinic of Soonchunhyang University Hospital, Korea. The subjects in the nonasthmatic control group had no history of broncho-pulmonary disease and had an FEV1>80% of predicted and an FEV1/FVC %>70%. The mild bronchial asthmatic group had an FEV1>70% and moderate bronchial asthmatic group had an FEV1<70%. All specimens were formalin-fixed, paraffin-embedded and processed for routine histological diagnosis. The study was approved by the ethics committee of Soonchunhyang University Hospital, and the patients provided written informed consent. The pattern of BLT2 expression in the bronchial biopsy specimens was detected immunohistochemically using an alkaline phosphatase substrate system.

### Example 9

### BLT2 Inhibition does not Interfere with Recruitment of T Lymphocytes into Airways

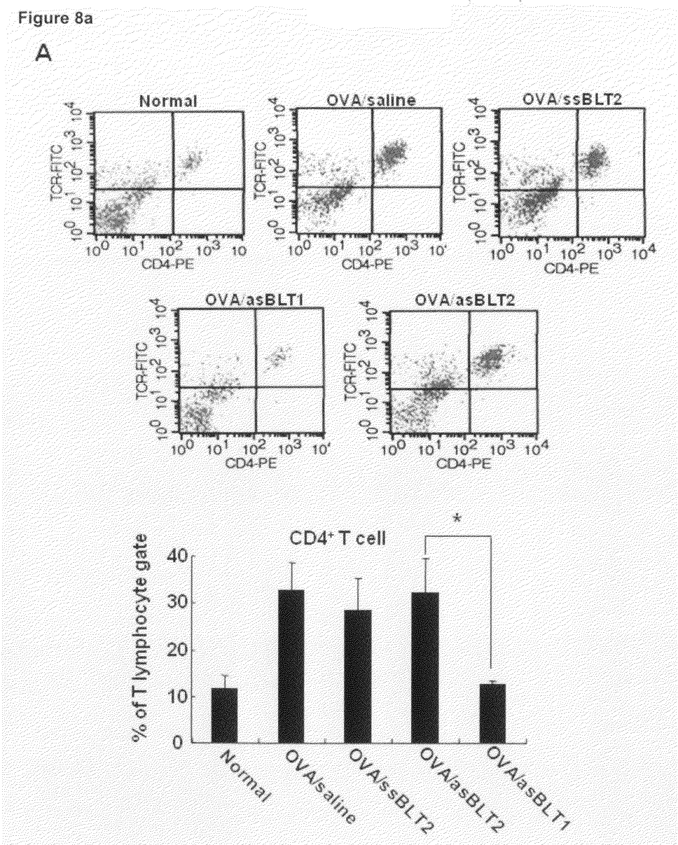

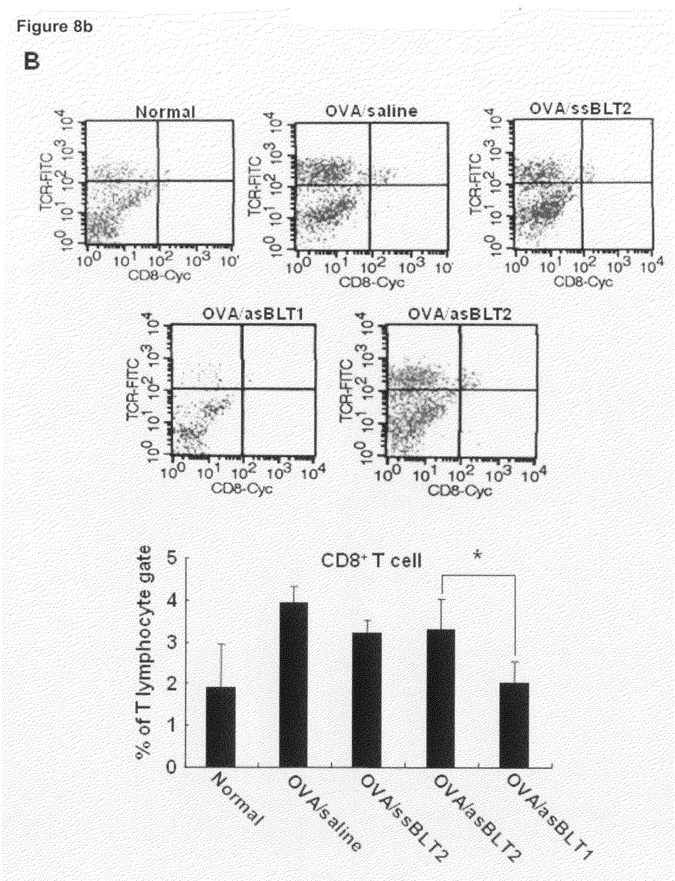

### 1) Flow Cytometric Analysis of Cells in BAL Fluid (FIGS. 8A-B)

For flow cytometric analysis, the cells in the BAL fluid were suspended in 50 μl of PBS containing 0.01% sodium azide and 0.1% BSA. BAL leukocytes were incubated for 30 min with 2.4G2 anti-FcγIII/II receptor (BD PharMingen) and stained for 30 min at 4° C. with FITC-conjugated anti-mouse TCR□chain (BD PharMingen) and PE-cy5 anti-mouse CD8a (BD PharMingen) or PE rat anti-mouse CD4 (BD PharMingen). Cytofluorimetry was performed with a FACS Calibur™ (Becton Dickinson, Franklin Lakes, N.J.), and the results were analyzed with CellQuest software (Becton-Dickinson).

### Example 10

### Antisense Rac Oligonucleotide Experiment

Oligonucleotides, from Genotech Co. (Korea), were synthesized with a phosphorothioate backbone to improve the resistance to endonuclease. The antisense oligonucleotide consisted of 17 nucleotides analogues to the 5′ end of the murine Rac mRNA sequence, which spans the translation initiation site. The control Rac oligonucleotide contained the same nucleotide composition as the antisense oligonucleotide. Oligonucleotides (1.25 mg/kg of weight) were injected into the tail veins of the mice 24 and 4 hr before OVA provocation. The sequences of the oligonucleotides used in this study were as follows. Control Rac: 5′-GATCAGTGCACACAGTG-3′ Antisense Rac: 5′-CACTTGATGGCCTGCAT-3′

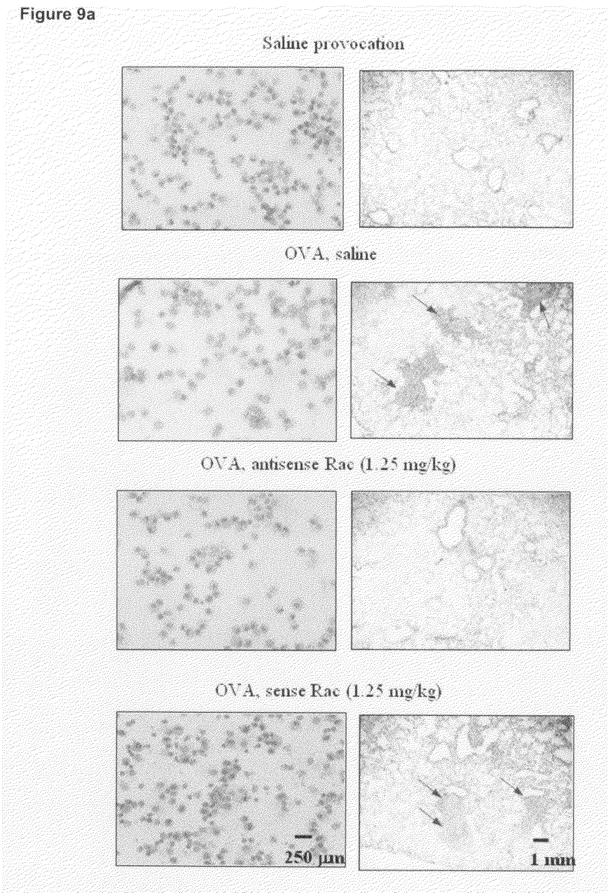

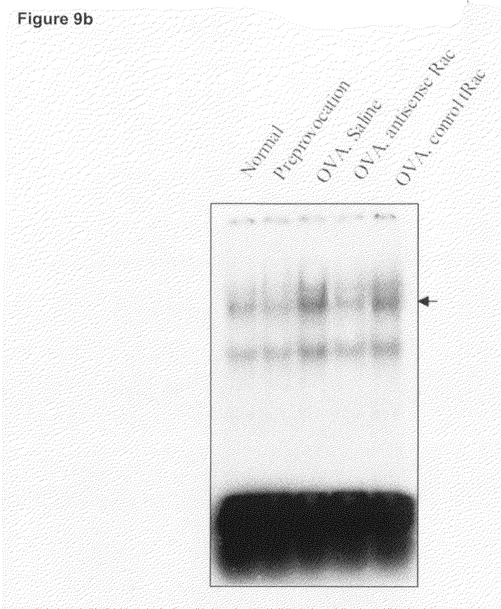

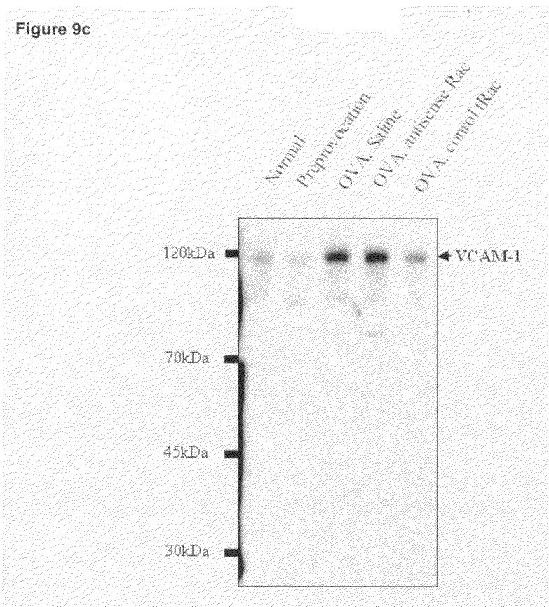

### 1) BAL and Histological Analysis of Lung (FIG. 9 A)

Inflammatory cells in the BAL fluid were collected by centrifugation (1,000 g for 3 min) and washed once in PBS. Cells were counted using a hemocytometer, and viability was assessed by trypan blue exclusion. In addition, cytospin was carried out for each BAL sample, which was then stained with Diff-Quick (Merck, Dorset, U.K.), enabling differential cell counts to be made. For histological analysis, the lungs of the mice were dissected 48 h after OVA challenge and fixed with 10% formaldehyde in PBS, dehydrated in ethanol-xylene, and embedded in paraffin. Multiple paraffin-embedded 6-μm sections were placed on 0.5% gelatin-coated slides, deparaffinized, and stained with hematoxylin-eosin (HE). Images were acquired using a BX51 microscope (Olympus, Tokyo, Japan) equipped with a DP71 digital camera (Olympus, Tokyo, Japan).

### 2) Electrophoretic Mobility Shift Assay (EMSA) (FIG. 9B)

A double-strand oligonucleotide corresponding to the consensus NF-κB binding motif and a mutant sequence were purchased from Santa Cruz Biotechnology Inc. (Santa Cruz, Calif.) and labeled with γ-32P-ATP using T4 polynucleotide kinase (Roche, Germany). Labeled oligonucleotide was then separated from free γ-32P-ATP on ProbeQuart™ G-50 microcolumns (Amersham Pharmacia Biotech, Ltd., UK) according to the manufacturer's protocol. Labeled oligonucleotide, 10 μg of nuclear extract and EMSA buffer were incubated for 1 h at room temperature in a final volume of 20 μl, after which the reaction mixture was subjected to electrophoresis.

### 3) SDS-PAGE and Immunoblot Analysis of Lung Cell Lysates (FIG. 9C)

Lung samples were prepared as described for preparation of total protein. The cell lysates were centrifuged at 13,000 g for 10 min and the supernatants subjected to SDS-PAGE on 10% acrylamide gels, followed by transfer to polyvinylidene difluoride (PVDF) membranes with a Novex wet transfer system (for 2 h at 100 V). The membranes were blocked for 1 h in Tris-buffered saline (TBS) containing 0.05% (v/v) Tween 20 plus 5% (w/v) nonfat dry skim milk, and then incubated for 2 h with the primary antibody in TBS containing 0.05% (v/v) Tween 20 plus 3% (w/v) BSA, followed for 1 h with horseradish peroxidase (HRP)-conjugated secondary antibody before development by enhanced chemiluminescence (ECL) (Amersham Pharmacia Biotech, Ltd., UK).

Result 1: Induction of BLT2 mRNA in the OVA-Induced Asthmatic Mouse Lung

To assess the role of BLT2 in OVA-induced allergic asthma, we measured levels of BLT2 and its ligand LTB4. As shown in FIG. 1A, LTB4 levels in BAL fluid increased following OVA challenge in this murine model of asthma, and peaked 48 h after the challenge. Semiquantitative RT-PCR analysis showed that levels of BLT2 mRNA in the lung increased dramatically after OVA challenge, while levels of BLT1 mRNA increased only slightly (FIG. 1B). To determine the distribution of BLT2 expression in the lung, we next carried out in situ hybridization with an antisense BLT2 probe and detected substantial elevation of BLT2 expression in the epithelium with some induction in the endothelium (FIG. 1C). Thus, both LTB4 and its receptor BLT2 appear to be upregulated in asthma.

### Result 2: Blockade of BLT2 Signaling Using a Pharmacological Antagonist or Antisense BLT2 Suppresses Airway Inflammation

To assess the possible mediatory role of BLT2 in asthmatic airway inflammation, a specific BLT2 antagonist, LY255283, was administrated intravenously 1 h before the 1% OVA challenge, and the mice were then sacrificed 48 h after challenge. Semiquantitative RT-PCR (FIG. 2A) or real-time PCR analysis (FIG. 2B) showed that the level of BLT2 mRNA in the lung was greatly elevated after the OVA challenge, and administration of LY255283 significantly reduced BLT2 levels. Histological analysis of the infiltration of inflammatory cells into the lung revealed increased airway obstruction and leukocyte infiltration following OVA challenge (FIGS. 2C & 2D); again, LY255283 diminished the effect (FIG. 2E). Quantitative analysis of the histological samples, which entailed grading the airway inflammation as described in Materials and Methods, revealed that LY255283 reduced the inflammation score by ˜66%, as compared to control (FIG. 2F).

To further evaluate the role of BLT2 in the pathogenesis of asthma, the effect of antisense BLT2 was analyzed. In this experiment, antisense BLT2 was administrated intravenously 24 h and 1 h before 10% OVA challenge, and the mice were sacrificed 48 h after challenge. Subsequent semiquantitative RT-PCR analysis showed that the antisense BLT2 reduced BLT2 expression in the lung without interfering with that of BLT1 (FIG. 3A). Similar results were also obtained with real-time PCR analysis (FIG. 3B). Airway eosinophil accumulation is a hallmark of asthmatic pulmonary inflammation, and we detected the accumulation of eosinophils in BAL fluid, which peaked 48 h after the 10% OVA challenge. Administration of antisense BLT2 reduced eosinophil infiltration in BAL fluids by ˜87%, whereas sense BLT2 had no inhibitory effect (FIG. 3C). In addition, histological analysis revealed increased airway obstruction and leukocyte infiltration following OVA challenge, and this effect, too, was diminished by antisense BLT2 (FIG. 3D). Consistent with those findings, administration of antisense BLT2 reduced inflammation scores by ˜67%, as compared to control.

### Result 3: Effect of BLT2 Inhibition on Airway AHR

To examine the contribution of BLT2 to AHR, we determined the increase in Penh (enhanced pause) elicited by methacholine (6.25-50 mg/ml). We found that OVA-challenged mice developed significant AHR to the inhaled methacholine, and
LY255283 or antisense BLT2 dramatically reduced Penh by ˜70% (based on area-under-the-curve calculations), which suggests that BLT2 is in some way critical for the AHR reaction (FIG. 4).

### Result 4: Attenuation of ROS Generation by BLT2 Inhibition

The ‘LTB4-BLT2’ cascade was previously shown to lead to enhanced ROS generation, which mediates various cellular effects (7). We therefore measured the ROS levels in BAL fluid following OVA challenge. As expected, the ROS levels in BAL fluid increased in the OVA-challenged mice. Notably, injection of antisense BLT2, but not sense BLT2, dramatically reduced ROS levels by ˜70% (FIG. 5A), suggesting ROS act as a mediator in the ‘LTB4-BLT2’ signaling leading to asthmatic symptoms. We also observed that administration of antisense BLT2 suppressed the level of LTB4 in BAL fluid by ˜72% (FIG. 5B), suggesting there may be cross-talk between LTB4 and BLT2, such that each affects the other. Indeed, similar instances of cross-talk between eicosanoid lipid ligands and their receptors have been described previously (30, 31).

### Result 5: Attenuation of NF-κB Activation by BLT2 Inhibition

ROS were previously reported to affect redox-sensitive factors such as NF-κB and AP-1 (32). To investigate the downstream signaling mechanism by which ‘LTB4-BLT2’ causes asthmatic symptoms in vivo, we used EMSA to assess NF-κB activation in the lungs of OVA-challenged mice. We tested nuclear extracts from lung tissue for their ability to bind a 32P-labeled oligonucleotide corresponding to the NF-κB consensus sequence. We found that OVA challenge elicited an increase in NF-κB binding activity, which was attenuated by prior administration of antisense BLT2 (FIG. 6A). Moreover, competition assays using excess unlabeled oligonucleotide (cold) confirmed that the binding was specific. NF-κB normally resides in the cytoplasm in an inactivated form complexed with IκB-α. Upon stimulation, IκB-α is rapidly phosphorylated and degraded, allowing NF-κB to translocate into the nucleus. As a further indication of NF κB activation, we analyzed the level of IκB-α following BLT2 inhibition. We detected substantial degradation of IκB-α following OVA challenge, but antisense BLT2 (FIG. 6B) or LY255283 (FIG. 6D) reduced that degradation by ˜50%. It is known that activation of NF-κB induces a variety of inflammatory genes, including adhesion molecules (e.g., VCAM-1) (33). We therefore also examined the effect of BLT2 blockade on levels of VCAM-1, which is regulated by NF-κB and is reportedly involved in eosinophil infiltration (34). As shown in FIGS. 6C and 6E, OVA challenge caused induction of VCAM-1 in lung tissue, and antisense BLT2 or LY255283 suppressed this effect by ˜60%.

Result 6: Enhanced Expression of BLT2 in Samples from Clinically Asthmatic Subjects

We next used immunohistochemical analysis to determine whether BLT2 levels are also elevated in human asthmatic subjects. Bronchial biopsy specimens were obtained from nonasthmatic controls (n=4) and patients with mild (n=4) or moderate (n=5) bronchial asthma. In accordance with the results obtained with the murine model of asthma (FIG. 1C), we found BLT2 expression to be significantly elevated in all mild and moderate bronchial asthma specimens. Representative bronchial specimens from asthma patients (D-F) show highly induced expression of BLT2, whereas those from healthy controls (A-C) do not (FIG. 7), which is suggestive of the potential role of BLT2 in the clinical pathogenesis of asthma. BLT2 expression was mainly elevated in the airway epithelial layers and microvascular endothelium in patient lung samples, which is similar to the pattern observed in the murine model of asthma.

Result 7: BLT2 Inhibition does not Interfere with Recruitment of T Lymphocytes into Airways

BLT1 was found to be responsible for early recruitment of CD4+ and CD8+ T cells into the airways in a model of allergic pulmonary inflammation, suggesting that the LTB4-BLT1 pathway is involved in linking early immune system activation and effector T cell recruitment (11). We asked whether BLT2 plays a similar role in T cell trafficking into airways. We found that significant numbers of CD4+ and CD8+ T cells were recruited into BAL fluid 12 h after OVA challenge (FIG. 8). Importantly, antisense BLT2 had no inhibitory effect on T cell trafficking into airways, while injection of antisense BLT1 markedly diminished this recruitment of CD4+ and CD8+ T cells by ˜93% and ˜95%, respectively. This suggests that the actions mediated by ‘LTB4-BLT2’ are quite distinct from those mediated by ‘LTB4-BLT1’ during the asthmatic response.

### Result 8: Antisense Rac Inhibit Airway Inflammation

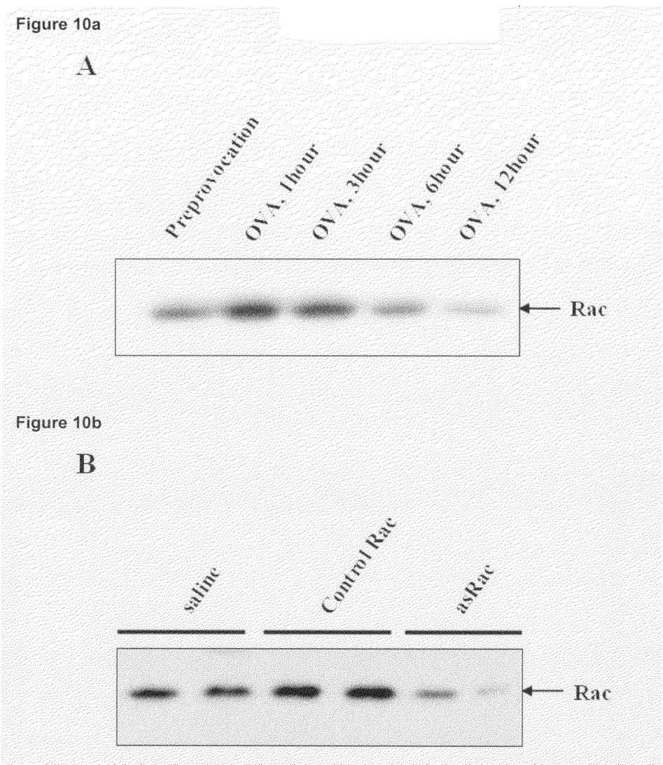

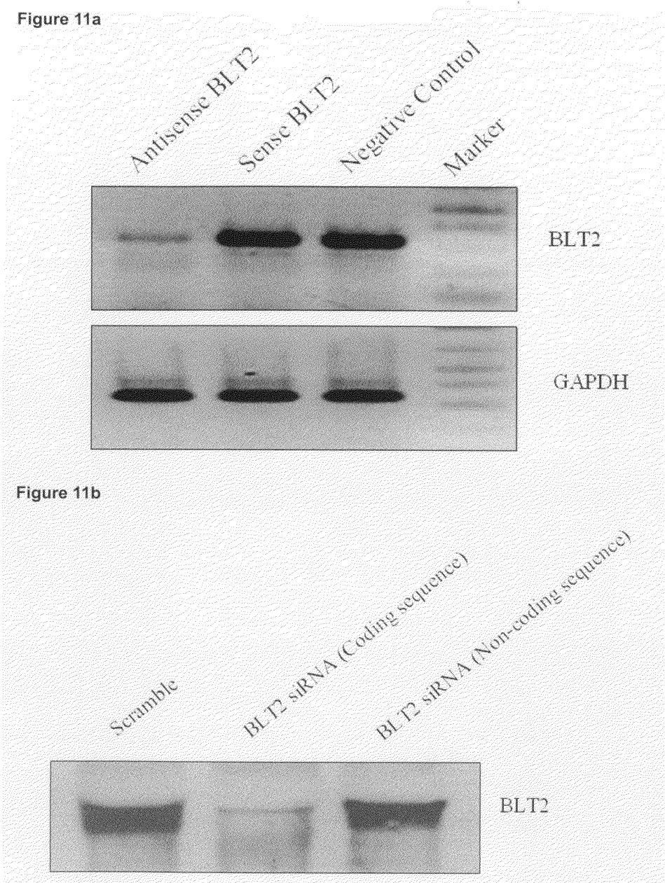

To examine the involvement of Rac in OVA-induced allergic inflammatory responses, we checked the activation of Rac by OVA provocation in lung lysate. It is well known that Rac translocates to the membrane from the cytosol when it is activated. Therefore, we prepared the membrane proteins from the lung tissues of OVA challenged mice and compared the amount of Rac in the membrane fraction as a marker of Rac activation. Rac was activated by OVA provocation in early time point (1 to 3 hr) and returned to normal at 6 hr after provocation (FIG. 10A).

Because there is no specific inhibitor for Rac, we designed antisense Rac oligonucleotide analogous to the 5′ end of murine Rac mRNA sequence, which spans the translation initiation site, to inhibit the endogenous expression of Rac. To confirm the effect of antisense Rac oligonucleotides on the expression of endogenous Rac, we injected the oligonucleotides into the tail veins of the mice and sacrificed after 12 hr. As expected, antisense Rac oligonucleotide inhibited the endogenous expression of Rac, while the control oligonucleotide didn't show significant effect on the expression of Rac in the lung tissues of the mice (FIG. 10B).

To assess the possible mediatory role of Rac in asthmatic airway inflammation, a specific Rac antisense, was administrated intravenously 24 h and 1 h before the 10% OVA challenge, and the mice were then sacrificed 48 h after challenge. Histological analysis of the infiltration of inflammatory cells into the lung revealed increased airway obstruction and leukocyte infiltration following OVA challenge (FIG. 9A); again, Rac antisense diminished the effect.

We tested nuclear extracts from lung tissue for their ability to bind a 32P-labeled oligonucleotide corresponding to the NF-κB consensus sequence. We found that OVA challenge elicited an increase in NF-κB binding activity, which was attenuated by prior administration of antisense Rac (FIG. 9B). Moreover, competition assays using excess unlabeled oligonucleotide (cold) confirmed that the binding was specific.

It is known that activation of NF-κB induces a variety of inflammatory genes, including adhesion molecules (e.g., VCAM-1) (33). We therefore also examined the effect of Rac blockade on levels of VCAM-1, which is regulated by NF-κB and is reportedly involved in eosinophil infiltration (34). As shown in FIG. 8C, OVA challenge caused induction of VCAM-1 in lung tissue, and antisense Rac suppressed this effect (FIG. 9C).

### Result 9: BLT2 Antisense Oligonucleotide Suppression Effect on BLT2 Expression

Suppressed BLT2 expression level was determined by RT-PCR. Rat2-BLT2 stable cells were plated at a density of 5×10⁴ cells/plate on 6 well plates. After 24 h, cells were transiently transfected with BLT2 specific antisense and sense oligonucleotide plasmid with Lipofectamin reagent and then incubated in fresh DMEM supplemented with 10% FBS for an additional 24 h. After additional incubation, the transfected cells were harvested for BLT2 transcripts analysis. Total RNA was reverse-transcribed and PCR amplify were performed with BLT2 forward primer: 5′ tctcatcgggcatcacaggt 3′ and reverse primer: 5′ ccaagctccacaccacgaag 3′. Non-transfected Rat2-BLT2 stable cells cDNA was used the negative control and GAPDH was shown as internal control. FIG. 9 shows the suppression effect of BLT2 antisense oligonucleotide on BLT2 expression level by RT-PCR. The result showed that the level of BLT2 mRNA was reduced by the antisense oligonucleotide, however the level of BLT2 mRNA was not affected by the sense oligonucleotide.

Result 10: BLT2 siRNA Suppression Effect on BLT2 Expression

BLT2 siRNA expression effect on BLT2 expression was addressed by Northern blotting. CHO-BLT2 stable cells were plated at a density of 1×10⁵ cells/plate on 60-mm dish. After 24 h, cells were transiently transfected with BLT2 specific siRNA, targeting for 1705-1724 bp in NM_{—}019839; 5′ GAAGGATGTCGGTCTGCTA 3′, with oligofectamin reagent and then incubated in fresh RPMI 1640 supplemented with 10% FBS for an additional 24 h. after additional incubation, total RNA was performed Northern blot with [32P]-dCTP labeled BLT2 probe. Scramble RNA and non-coding sequence BLT2 siRNA were used the negative control. A 110 bp PCR fragment was amplified with pcDNA3.1-BLT2 clone using the following two primers, forward primer: 5′ cttctcatcgggcatcacag 3′ and reverse primer: 5′ atccttctgggcctacaggt 3′. This probe was located mainly in the BLT2 coding region. Total RNA was extracted with TRIzol reagent and then loaded the ten microgram total RNA for 2 h in MOPS containing agarose gel. After this step, the total RNA was transferred the Hybond N⁺ membrane for overnight with 20×SSC buffer. The membrane was hybridized with [32P]-dCTP labled BLT2 probe in the hybridization buffer for 18 h at 68□. And then, washed in 0.1×SSC (0.1% SDS) for 1 h at 68□ and subjected to autoradiography. FIG. 10 shows the suppression effect of BLT2 siRNA on BLT2 expression level by Northern blot. The result showed that the level of BLT2 mRNA was reduced by the BLT2 siRNA (coding sequence), however the level of BLT2 mRNA was not affected by the BLT2 siRNA (non-coding sequence).

As disclosed above, the present inventors investigated the role of BLT2 in the pathogenesis of asthma using a murine model and demonstrated that BLT2 plays a critical role in the development of AHR and airway inflammation by employing BLT2 inhibitors, such as antisense oligonucleotide. Therefore, the BLT 2 inhibitors according to the present invention can be effectively used as a therapeutic composition for treating asthma.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

### REFERENCES

1. Lewis R A., Austen K F, Soberman R J (1990) N Engl J Med 323:645-655.
2. Samuelsson B, Dahlen S E, Lindgren J A., Rouzer C A, Serhan C N (1987) Science 237:1171-1176.
3. Yokomizo T, Izumi T, Shimizu T (2001) Arch Biochem Biophys 385:231-241.
4. Serhan C N (1996) Nature 384:23-24.
5. Gaudreault E, Thompson C, Stankova J, Rola-Pleszczynski M (2005) J Immunol 174:3617-3625.
6. Lindsay M A, Perkins R^{S}, Barnes P J, Giembycz M A (1998) J Immunol 160:4526-4534.
7. Woo C H, You H J, Cho S H, Eom Y W, Chun J S, Yoo Y J, Kim J H (2002) J Biol Chem 277:8572-8578.
8. Tager A M, Dufour J H, Goodarzi K, Bercury S D, von Andrian U H, Luster A D (2000) J Exp Med 192:439-446.
9. Silbaugh S A, Stengel P W, Williams G D, Herron D K, Gallagher P, Baker S R (1987) Am Rev Respir Dis 136:930-934.
10. Goodarzi K, Goodarzi M, Tager A M, Luster A D, von Andrian U H (2003) Nat Immunol 4:965-973.
11. Tager A M, Bromley S K, Medoff B D, Islam S A, Bercury S D, Friedrich E B, Carafone A D, Gerszten R E, Luster A D (2003) Nat Immunol 4:982-990.
12. Ott V L, Cambier J C, Kappler J, Marrack P, Swanson B J (2003) Nat Immunol 4:974-981.
13. Gelfand E W, Dakhama A (2006) J Allergy Clin Immunol 117:577-582.
14. Taube C, Miyahara N, Ott V, Swanson B, Takeda K, Loader J, Shultz L D, Tager A M, Luster A D, DakhamaA, et al (2006) J Immunol 176:3157-3164.
15. Islam S A, Thomas S Y, Hess C, Medoff B D, Means T K, Brander C, Lilly C M, Tager A M, Luster A D (2006) Blood 107:444-453.
16. Steiner D R, Gonzalez N C, Wood J G (2001) J Appl Physiol 91:1160-1167.
17. Turner C R, Breslow R, Conklyn M J, Andresen C J, Patterson D K, Lopez-Anaya A, Owens B, Lee P, Watson J W, Showell H J (1996) J Clin Invest 97:381-367.
18. Montuschi P, Barnes P J (2002) J Allergy Clin Immunol 109:615-620.
19. Shindo K, Koide K, Fukumura M (1997) Thorax 52:1024-1029.
20. Henderson W R, Jr Lwis D B, Albert R K, Zhang Y, Lamm W J, Chiang G K, Jones F, Eriksen P, Tien Y T, Jonas M, et al (1996) J Exp Med 184:1483-1494.
21. Kamohara M, Takasaki J, Matsumoto M, Saito T, Ohishi T, Ishii H, Furuichi K (2000) Biol Chem 275:27000-27004.
22. Yokomizo T, Izumi T, Chang K, Takuwa, Shimizu T (1997) Nature 387:620-624.
23. Yokomizo T, Kato K, Terawaki K, Izumi T, Shimizu T (2000) J Exp Med 192:421-432.
24. Qiu H, Johansson A S, Sjostrom M, Wan M, Schroder 0, Palmblad J, Haeggstrom J Z (2006) Proc Natl Acad Sci USA 103:6913-6918.
25. Terawaki K, Yokomizo T, Nagase T, Toda A, Taniguchi M, Hashizume K, Yagi T, Shimizu T (2005) J Immunol 175:4217-4225.
26. Miyahara N, Takeda K, Miyahara S, Taube C, Joetham A, Koya T, Matsubara S, Dakhama A, Tager A M, Luster A D, et al (2005) J Immunol 174:4979-4984.
27. Miyahara N, Takeda K, Miyahara S, Matsubara S, Koya T, Joetham A, Krishnan E, Dakhama A, Haribabu B, Gelfand E W (2005) Am J Respir Crit Care Med 172:161-167.
28. Kanehiro A, Ikemura T, Mäkela MJ, Lahn M, Joetham A, Dakhama A, Gelfand E W (2001) Am J Respir Crit Care Med 163:173-184.
29. Cho S H, You H J, Woo C H, Yoo Y J, Kim J H (2004) J Immunol 173:624-631.
30. Lee Y C, Lee K S, Park S J, Park H S, Lim J S, Park K H, Im M J, Choi I W, Lee H K, Kim U H (2004) Faseb J 18:1917-1919.
31. Dohadwala M, Batra R K, Luo J, Lin Y. Krysan K, Pold M, Sharma S, Dubinett S M (2002) J Biol Chem 277:50828-50833.
32. Yoo M H, Song H, Woo C H, Kim H, Kim J H (2004) Oncogene 23:9259-9268.
33. Henderson W R, Jr Chi E Y, Teo J L, Nguyen C, Kahn M (2002) J Immunol 169:5294-5299.
34. Wilson S J, Wallin A, Della-Cioppa Q Sandstrom T, Holgate S T (2001) Am J Respir Crit Care Med 164:1047-1052.
35. Huang W W, Garcia-Zepeda E A, Sauty A, Oettgen H C, Rothenberg M E, Luster A D (1998) J Exp Med 188:1063-1074.
36. Miyahara N, Miyahara S, Takeda K, Gelfand E W (2006) Allergol Int 55:91-97.
37. Woo C H, Lim J H, Kim J H (2005) Am J Physiol Lung Cell Mol Physiol 288:L307-L316.
38. Luster A D, Tager A M (2004) Nat Rev Immunol 4:711-724.
39. Boldogh I, Bacsi A, Choudhury B K, Dharajiya N, Alam R, Hazra T K, Mitra S, Goldblum R M, Sur S (2005) J Clin Invest 115:2169-2179.
40. Cortijo J, Marti-Cabrera M, de la Asuncion J G Pallardo F V, Esteras A, Bruseghini L, Vina J, Morcillo E J (1999) Free Radic Biol Med 27:392-400.
41. MacNee W (2001) Eur J Pharmacol 429:195-207.
42. Dworski R (2000) Thorax 55 Suppl 2:S51-S53.
43. Lee S H, Seo M J, Choi S M, Sohn Y S, Kang K K, Ahn B O, Kwon J W, Yoo M (2005) Arch Pharm Res 28:1350-1357.
44. Barnes P J, Karin M (1997) N Engl J Med 336:1066-1071.
45. Hamilton L M, Davies D E, Wilson S J, Kimber I, Dearman R J, Holgatem S T (2001) Monaldi Arch Chest Dis 56:48-54.
46. Hart L A, Krishnan V L, Adcock I M, Barnes P J, Chung K F (1998) Am J Respir Crit Care Med 158:1585-1592.
47. Shao W H, Del Prete A, Bock C B, Haribabu B (2006) J Immunol 176:6254-6261.

1-25. (canceled)

26. A method for treating a patient with asthma, which comprises administering a therapeutically effective amount of a substance that inhibits the expression or intracellular signaling of BLT2 to the patient

27. The method according to claim 26, wherein the substance is a compound that binds to BLT2 and inhibits the intracellular signaling of BLT2.

28. The method according to claim 27, wherein the compound is LY255283 (1-[5-ethyl-2-hydroxy-4-[[6-methyl-6-(1H-tetrazol-5-yl)heptyl]oxy]phenyl]-ethanone).

29. The method according to claim 26, wherein the substance is an antibody to BLT2 that inhibits the intracellular signaling of BLT2.

30. The method according to claim 26, wherein the substance is an antisense or siRNA oligonucleotide that inhibits the expression of BLT2.

31. The method according to claim 30, wherein the antisense oligonucleotide has a base sequence of SEQ ID NO: 6.

32. The method according to claim 30, wherein the siRNA oligonucleotide has a sense sequence of SEQ ID NO: 7 and an antisense sequence of SEQ ID NO: 8.

33. The method according to claim 26, wherein the substance is a compound that inhibits the upstream or downstream signaling pathway of BLT2.

34. The method according to claim 26, wherein the asthma is characterized in that BLT2 protein is over-expressed in the lung airway.

35. The method according to claim 34, wherein the BLT2 activation causes asthmatic symptoms by elevating ROS generation and subsequent NF-κB activation.

36. The method according to claim 26, wherein the treatment of asthma is accomplished by reducing eosinophil infiltration into lung airway, airway inflammation and airway hyperresponsiveness (AHR).

37. The method according to claim 26, wherein the substance is combined with other anti-asthma drugs.

38. A pharmaceutical composition for the treatment of asthma, which comprises a substance that inhibits the expression or intracellular signaling of BLT2 as an active ingredient.

39. A method for screening a substance for treating asthma, which comprises the steps of:
(a) contacting the substance to be analyzed to a cell containing BLT2 gene or protein; and,
(b) measuring the expression or intracellular signaling level of BLT2, wherein if the expression or intracellular signaling level of is down-regulated, the substance is determined to have a potency to treat asthma.

40. A kit for detecting asthma, which comprises a primer or probe having a base sequence complementary to the base sequence of BLT2 gene.

41. The kit according to claim 40, which comprises a pair of primers having base sequences of SEQ ID NOs: 2 and 3.

42. A kit for detecting asthma, which comprises an antibody binding specifically to BLT2 protein.


SEQUENCE LISTING (14 sequences)

SEQ ID NO: 1 - DNA, 2798 bp, homo sapiens
aaactggccctggccctgaaccaaataccttgaaccctcgtaaactccataccctgacccccttgttttggatatacccaggtagaacaactctctctcactgtctgttgtgaggatacgctgtagcccactcattaagtacattctcctaataaatgctttggactgatcaccctgccagtcttttgtcttgggcaatctatacttttctcagaggttcccaaggcctactgaagggacttaacatactcttaatggctttcctctctcttgttttaccttatgccctcacttcctgagttaacctcccaaatacaggatcacctgtacccaagcccttagctcaagaatacaggatcacctgtacccaagcccttagctcaagctctgctttggaagaacccaaactaagacagtgctcctggtgccctccccaagcaacctcaagttctggctgttacttgagcagaggcctttcttttcccttcccccagctctatccatctgccaggcccccctcaaatctcttcatttccaagttttgcttgacttttccaagaggagagggctgcttcttagtatgtccctactcatcctttcctttcttgtcttgtatcctggtgcagcctggtaatggggcctcttcatggttgtgtgtcatgactccctaaccattatgcctccatgcatcccctgttcctcctggaacctagcaccatgccttacatggaaaagctgtcattgacagcccggtgagagccctgagggtggagtgactggggcagggcctgaggcaagaggtgggaggaggtaggaggccaggggctcagccggaccaggagactggaaacaggcaaggataaggcaggtgggggactgagttgtttgggtcacctctgcaggccagagagaccaggcaacatacacactgcagaaggtgggctgggaggattggggccagagctgggggagggatgagaacagaagcaggaccaggattcagcagagtcctcctatttccttccaccaccagggaatcttactgccccacttcagcttgtgctgtttcctggcaaggcaggctctcacatgcctggacgcctgggtgcgttggtgatgggaaggagcagggtgagggaggggccccaggagaggcccaggatgagcctcatcttgtccctccccattcttgtcttaccctctgcaaatgtgataggcacaggacaggagtaggcacctcgcctactgctgcttaacctttcagcttctccaggcccccaatcctgcttgctcccagcttggtaagtagatctgtgcacgtccctttacaccccaccatccagttttgcccagatgtgctagaatggggctggacaaagaaggaggggccagactagaggagtggtggtagagatagtgacagcctggggtgaggactttatgcctgtttaccactgagctctgggaaggaggccaggagtggggcaggtcaactgactgggagcaggggatctgggttccaagaaggagttgtgtttgaggtggggtctgggtcctcgtggaagtcaggactcccaggcagaaaagaggcaggctgcagggaagtaaggaggaggcatggcaccttctcatcgggcatcacaggtggggttttgccccacccctgaacgccctctgtggcgccttccacccacctgtaggcccagaaggatgtcggtctgctaccgtcccccagggaacgagacactgctgagctggaagacttcgcgggccacaggcacagccttcctgctgctggcggcgctgctggggctgcctggcaacggcttcgtggtgtggagcttggcgggctggcggcctgcacgggggcgaccgctggcggccacgcttgtgctgcacctggcgctggccgacggcgcggtgctgctgctcacgccgctctttgtggccttcctgacccggcaggcctggccgctgggccaggcgggctgcaaggcggtgtactacgtgtgcgcgctcagcatgtacgccagcgtgctgctcaccggcctgctcagcctgcagcgctgcctcgcagtcacccgccccttcctggcgcctcggctgcgcagcccggccctggcccgccgcctgctgctggcggtctggctggccgccctgttgctcgccgtcccggccgccgtctaccgccacctgtggagggaccgcgtatgccagctgtgccacccgtcgccggtccacgccgccgcccacctgagcctggagactctgaccgctttcgtgcttcctttcgggctgatgctcggctgctacagcgtgacgctggcacggctgcggggcgcccgctggggctccgggcggcacggggcgcgggtgggccggctggtgagcgccatcgtgcttgccttcggcttgctctgggccccctaccacgcagtcaaccttctgcaggcggtcgcagcgctggctccaccggaaggggccttggcgaagctgggcggagccggccaggcggcgcgagcgggaactacggccttggccttcttcagttctagcgtcaacccggtgctctacgtcttcaccgctggagatctgctgccccgggcaggtccccgtttcctcacgcggctcttcgaaggctctggggaggcccgagggggcggccgctctagggaagggaccatggagctccgaactacccctcagctgaaagtggtggggcagggccgcggcaatggagacccggggggtgggatggagaaggacggtccggaatgggacctttgacagcagaccct

SEQ ID NO: 2 - DNA, 1170 bp, homo sapiens
  CDS (1)..(1167)
atggcaccttctcatcgggcatcacaggtggggttttgccccacccctgaacgccctctgtggcgccttccacccacctgtaggcccagaaggatgtcggtctgctaccgtcccccagggaacgagacactgctgagctggaagacttcgcgggccacaggcacagccttcctgctgctggcggcgctgctggggctgcctggcaacggcttcgtggtgtggagcttggcgggctggcggcctgcacgggggcgaccgctggcggccacgcttgtgctgcacctggcgctggccgacggcgcggtgctgctgctcacgccgctctttgtggccttcctgacccggcaggcctggccgctgggccaggcgggctgcaaggcggtgtactacgtgtgcgcgctcagcatgtacgccagcgtgctgctcaccggcctgctcagcctgcagcgctgcctcgcagtcacccgccccttcctggcgcctcggctgcgcagcccggccctggcccgccgcctgctgctggcggtctggctggccgccctgttgctcgccgtcccggccgccgtctaccgccacctgtggagggaccgcgtatgccagctgtgccacccgtcgccggtccacgccgccgcccacctgagcctggagactctgaccgctttcgtgcttcctttcgggctgatgctcggctgctacagcgtgacgctggcacggctgcggggcgcccgctggggctccgggcggcacggggcgcgggtgggccggctggtgagcgccatcgtgcttgccttcggcttgctctgggccccctaccacgcagtcaaccttctgcaggcggtcgcagcgctggctccaccggaaggggccttggcgaagctgggcggagccggccaggcggcgcgagcgggaactacggccttggccttcttcagttctagcgtcaacccggtgctctacgtcttcaccgctggagatctgctgccccgggcaggtccccgtttcctcacgcggctcttcgaaggctctggggaggcccgagggggcggccgctctagggaagggaccatggagctccgaactacccctcagctgaaagtggtggggcagggccgcggcaatggagacccggggggtgggatggagaaggacggtccggaatgggacctttga

SEQ ID NO: 3 - protein, 389 aa, homo sapiens
MAPSHRASQVGFCPTPERPLWRLPPTCRPRRMSVCYRPPGNETLLSWKTSRATGTAFLLLAALLGLPGNGFVVWSLAGWRPARGRPLAATLVLHLALADGAVLLLTPLFVAFLTRQAWPLGQAGCKAVYYVCALSMYASVLLTGLLSLQRCLAVTRPFLAPRLRSPALARRLLLAVWLAALLLAVPAAVYRHLWRDRVCQLCHPSPVHAAAHLSLETLTAFVLPFGLMLGCYSVTLARLRGARWGSGRHGARVGRLVSAIVLAFGLLWAPYHAVNLLQAVAALAPPEGALAKLGGAGQAARAGTTALAFFSSSVNPVLYVFTAGDLLPRAGPRFLTRLFEGSGEARGGGRSREGTMELRTTPQLKVVGQGRGNGDPGGGMEKDGPEWDL

SEQ ID NO: 4 - DNA, 1077 bp, homo sapiens
  CDS (1)..(1074)
atgtcggtctgctaccgtcccccagggaacgagacactgctgagctggaagacttcgcgggccacaggcacagccttcctgctgctggcggcgctgctggggctgcctggcaacggcttcgtggtgtggagcttggcgggctggcggcctgcacgggggcgaccgctggcggccacgcttgtgctgcacctggcgctggccgacggcgcggtgctgctgctcacgccgctctttgtggccttcctgacccggcaggcctggccgctgggccaggcgggctgcaaggcggtgtactacgtgtgcgcgctcagcatgtacgccagcgtgctgctcaccggcctgctcagcctgcagcgctgcctcgcagtcacccgccccttcctggcgcctcggctgcgcagcccggccctggcccgccgcctgctgctggcggtctggctggccgccctgttgctcgccgtcccggccgccgtctaccgccacctgtggagggaccgcgtatgccagctgtgccacccgtcgccggtccacgccgccgcccacctgagcctggagactctgaccgctttcgtgcttcctttcgggctgatgctcggctgctacagcgtgacgctggcacggctgcggggcgcccgctggggctccgggcggcacggggcgcgggtgggccggctggtgagcgccatcgtgcttgccttcggcttgctctgggccccctaccacgcagtcaaccttctgcaggcggtcgcagcgctggctccaccggaaggggccttggcgaagctgggcggagccggccaggcggcgcgagcgggaactacggccttggccttcttcagttctagcgtcaacccggtgctctacgtcttcaccgctggagatctgctgccccgggcaggtccccgtttcctcacgcggctcttcgaaggctctggggaggcccgagggggcggccgctctagggaagggaccatggagctccgaactacccctcagctgaaagtggtggggcagggccgcggcaatggagacccggggggtgggatggagaaggacggtccggaatgggacctttga

SEQ ID NO: 5 - protein, 358 aa, homo sapiens
MSVCYRPPGNETLLSWKTSRATGTAFLLLAALLGLPGNGFVVWSLAGWRPARGRPLAATLVLHLALADGAVLLLTPLFVAFLTRQAWPLGQAGCKAVYYVCALSMYASVLLTGLLSLQRCLAVTRPFLAPRLRSPALARRLLLAVWLAALLLAVPAAVYRHLWRDRVCQLCHPSPVHAAAHLSLETLTAFVLPFGLMLGCYSVTLARLRGARWGSGRHGARVGRLVSAIVLAFGLLWAPYHAVNLLQAVAALAPPEGALAKLGGAGQAARAGTTALAFFSSSVNPVLYVFTAGDLLPRAGPRFLTRLFEGSGEARGGGRSREGTMELRTTPQLKVVGQGRGNGDPGGGMEKDGPEWDL

SEQ ID NO: 6 - DNA, 15 bp, Artificial Sequence
  hBLT2 anti-sense sequence
cagcagtgtctcgtt

SEQ ID NO: 7 - RNA, 21 nt, Artificial Sequence
  hBLT2 siRNA sense sequence
gaaggaugucggucugcuauu

SEQ ID NO: 8 - RNA, 21 nt, Artificial Sequence
  hBLT2 siRNA antisense sequence
uagcagaccgacauccuucuu

SEQ ID NO: 9 - DNA, 25 bp, Artificial Sequence
  forward primer for hBLT2
agcctggagactctgaccgctttcg

SEQ ID NO: 10 - DNA, 25 bp, Artificial Sequence
  reverse primer for hBLT2
gacgtagagcaccgggttgacgcta

SEQ ID NO: 11 - DNA, 20 bp, Artificial Sequence
  forward primer for re-hBLT2
tctcatcgggcatcacaggt

SEQ ID NO: 12 - DNA, 20 bp, Artificial Sequence
  reverse primer for re-hBLT2
ccaagctccacaccacgaag

SEQ ID NO: 13 - DNA, 2398 bp, homo sapiens
  CDS (242)..(874)
gggaggccggatgtgagtggagcggccatttcctgtttctctgcagttttcctcagctttgggtggtggccgctgccgggcatcggcttccagtccgcggagggcgaggcggcgtggacagcggccccggcacccagcgccccgccgcccgcaagccgcgcgcccgtccgccgcgccccgagcccgccgcttcctatctcagcgccctgccgccgccgccgcggcccagcgagcggccctgatgcaggccatcaagtgtgtggtggtgggagacggagctgtaggtaaaacttgcctactgatcagttacacaaccaatgcatttcctggagaatatatccctactgtctttgacaattattctgccaatgttatggtagatggaaaaccggtgaatctgggcttatgggatacagctggacaagaagattatgacagattacgccccctatcctatccgcaaacagttggagaaacgtacggtaaggatataacctcccggggcaaagacaagccgattgccgatgtgttcttaatttgcttttcccttgtgagtcctgcatcatttgaaaatgtccgtgcaaagtggtatcctgaggtgcggcaccactgtcccaacactcccatcatcctagtgggaactaaacttgatcttagggatgataaagacacgatcgagaaactgaaggagaagaagctgactcccatcacctatccgcagggtctagccatggctaaggagattggtgctgtaaaatacctggagtgctcggcgctcacacagcgaggcctcaagacagtgtttgacgaagcgatccgagcagtcctctgcccgcctcccgtgaagaagaggaagagaaaatgcctgctgttgtaaatgtctcagcccctcgttcttggtcctgtcccttggaacctttgtacgctttgctcaaaaaaaaacaaaaaaaaaaaacaaaaaaaaaaaacaacggtggagccttcgcactcaatgccaactttttgttacagattaatttttccataaaaccattttttgaaccaatcagtaattttaaggttttgtttgttctaaatgtaagagttcagactcacattctattaaaatttagccctaaaatgacaagccttcttaaagccttatttttcaaaagcgccccccccattcttgttcagattaagagttgccaaaataccttctgaactacactgcattgttgtgccgagaacaccgagcactgaactttgcaaagaccttcgtctttgagaagacggtagcttctgcagttaggaggtgcagacacttgctctcctatgtagttctcagatgcgtaaagcagaacagcctcccgaatgaagcgttgccattgaactcaccagtgagttagcagcacgtgttcccgacataacattgtactgtaatggagtgagcgtagcagctcagctctttggatcagtctttgtgatttcatagcgagttttctgaccagcttttgcggagattttgaacagaactgctatttcctctaatgaagaattctgtttagctgtgggtgtgccgggtggggtgtgtgtgatcaaaggacaaagacagtattttgacaaaatacgaagtggagatttacactacattgtacaaggaatgaaagtgtcacgggtaaaaactctaaaaggttaatttctgtcaaatgcagtagatgatgaaagaaaggttggtattatcaggaaatgttttcttaagcttttcctttctcttacacctgccatgcctccccaaattgggcatttaattcatctttaaactggttgttctgttagtcgctaacttagtaagtgcttttcttatagaaccccttctgactgagcaatatgcctccttgtattataaaatctttctgataatgcattagaaggtttttttgtcgattagtaaaagtgctttccatgttactttattcagagctaataagtgctttccttagttttctagtaactaggtgtaaaaatcatgtgttgcagctttatagtttttaaaatattttagataattcttaaactatgaaccttcttaacatcactgtcttgccagattaccgacactgtcacttgaccaatactgaccctctttacctcgcccacgcggacacacgcctcctgtagtcgctttgcctattgatgttcctttgggtctgtgaggttctgtaaactgtgctagtgctgacgatgttctgtacaacttaactcactggcgagaatacagcgtgggacccttcagccactacaacagaattttttaaattgacagttgcagaattgtggagtgtttttacattgatcttttgctaatgcaattagcattatgttttgcatgtatgacttaataaatccttgaatcata

SEQ ID NO: 14 - protein, 211 aa, homo sapiens
MQAIKCVVVGDGAVGKTCLLISYTTNAFPGEYIPTVFDNYSANVMVDGKPVNLGLWDTAGQEDYDRLRPLSYPQTVGETYGKDITSRGKDKPIADVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVLCPPPVKKRKRKCLLL